(19) United States
(12) Patent Application Publication
(10) Pub. No.: US 2002/0192159 A1
(43) Pub. Date: Dec. 19, 2002

(54) Single-patient drug trials used with accumulated database: flowchart

(76) Inventor: Donald P. Reitberg, Bedminster, NJ (US)

Correspondence Address: DAVIDSON, DAVIDSON & KAPPEL, LLC, 485 SEVENTH AVENUE, 14TH FLOOR, NEW YORK, NY, 10018, US

(21) Appl. No.: 09/872,815

(22) Filed: Jun. 1, 2001

(51) Int. Cl. A61K 49/00; G06F 17/60

(52) U.S. Cl. 424/9.1; 705/3

(57) ABSTRACT

A method of evaluating and/or optimizing clinical outcomes and providing rational pharmacotherapy in an individual or animal requiring chronic drug therapy is provided.

### BACKGROUND OF THE INVENTION

The present invention relates to improving the treatment of chronic illness and conditions in humans and animals. In particular, the invention relates to kits and methods that improve chronic treatments using data obtained from individual randomized, crossover, parallel, (n=1 or single patient) open-label, single-blind or double-blind studies.

Inappropriate prescribing of potent and potentially dangerous drugs is a problem of staggering dimensions. Nonetheless, no commercial solution has been advanced to ensure appropriate treatment. Presently, doctors prescribe medications which have approved indications determined by large clinical trials. Drug manufacturers also demonstrate a product's safety and effectiveness using well controlled clinical studies in populations likely to require its use (e.g. hypertensive patients for antihypertensive drugs). Relatively small numbers of highly selected subjects are utilized. Too often, these studies do not accurately predict the safety and effectiveness of a medication for individuals actually treated in practice.

Thus, prescribers are at a disadvantage because a highly selected, often homogeneous group of patients is actually studied for marketing approval. The prescribing physician often cannot distinguish which drugs are safe and effective for his/her heterogeneous population of individual patients. Even in homogeneous groups of patients, individual variation is usually large when a pharmaceutical company measures a drug's disposition and activity. Therefore, average results may be poorly suited to the needs of any given individual. It is rarely clear to the prescribing physician how an individual patient might respond to a given medication. This is because all people respond differently, both positively and negatively, based upon individual genetic and environmental factors.

Furthermore, the physician rarely has objective information to help decide between alternative therapies for an individual patient. Although the physician wants unbiased data concerning how a patient responds to a given therapy, such data is almost never available unless the patient is in a drug trial. The physician is almost always required to use subjective “clinical judgement.”

Pharmaceutical manufacturers are also at a disadvantage since they have no means of providing unbiased data for individual patients. Manufacturers rarely receive feedback on how a drug is used in actual practice unless an adverse event is reported. Other organizations, e.g., FDA, HMOs, often need unbiased information for regulatory, patient care and business purposes. Currently, unreliable retrospective databases, such as government or health maintenance organizations' epidemiologic databases, are often used.

Group clinical trials of the type conducted for governmental regulatory approval, such as U.S. Food and Drug Administration (“FDA”) approval of a new treatment are intended to demonstrate that, on the average, the new treatment is superior to placebo. However, group prospective effectiveness studies are of a parallel design, and do not address a specific patient's response to drug compared to another drug or placebo control.

Results from group clinical trials are generalizations that provide an appropriate basis for regulatory decisions, but do not necessarily apply to specific subgroups or individuals. The FDA is well aware that “one size does not fit all”, as indicated by the following excerpt from the 1988 Guideline for the Format and Content of the Clinical and Statistical Sections of an Application:

“If the size of the study permits, relevant demographic or baseline value-defined subgroups should be examined for unusually large or small responses and the results presented, e.g., comparison of effects by severity groups, by age, sex, or race, or by history of prior treatment with a drug of the same class.”

Ideally, the FDA would like to be able to predict which patients will respond positively to the drug. However, the clinical trials conducted by pharmaceutical companies are rarely sufficient for this purpose. Therefore, in the end, responsibility for the evaluation of whether a drug is safe and effective for a specific patient lies with the practicing physician. The mere fact of FDA approval, based on the result of group clinical trials, only suggests that the drug might be safe and effective in that patient.

In 1985, investigators proposed a single-patient drug trial as a possible solution to the above-identified problems. Using a single-patient study design, e.g., a patient is treated with a medication and a placebo in a double-blind randomized manner (referred to in the art as an “n of 1” or “n=1” or single patient drug trial). This approach permits assessment of whether a medication regimen is appropriate for an individual patient in terms of medical benefit and harm, and eliminates patient/physician bias by making the medication and placebo look and/or taste the same. Thus, a toxic or ineffective treatment can be avoided using objective criteria and new treatment regimens can be pursued for well documented reasons and similarly tested, if needed.

The single-patient method has significant shortcomings. It has failed to provide validated results. There was no appreciation that the data obtained from the single patient trial should be compared against a database compiled from similarly affected and tested patients. Moreover, no guidance was provided concerning therapeutic alternatives or generic equivalents based upon a database comprised of earlier patient experience during single-patient, parallel or control group trials.

No commercial products or methodology are believed to be available which allow objective and definitive measurement of individual patient compatibility with drug treatment compared to placebo, a therapeutic alternative, a different dose of a drug or a generic equivalent. The present invention addresses this need.

### OBJECTS AND SUMMARY OF THE INVENTION

It is an object of the invention to provide a method for managing health care costs.

It is another object of the invention to provide methods and kits which can assess the appropriateness of specific drug treatment in individuals, particularly those suffering from chronic illnesses or conditions.

It is a further object to provide methods and kits for testing therapeutic alternatives for drug treatments in individuals.

It is another object of the invention is to provide methods and kits for verifying generic equivalence of known medications.

It is another object of the invention is to provide methods and kits to optimize clinical outcomes, providing rational (evidence-based) pharmacotherapy and decrease health care costs.

It is another object of the present invention to develop a method of evaluating the therapeutic response of individual human patients to chronic therapy with a drug.

It is another object of the present invention to provide a method for doing business for optimizing clinical outcomes, providing rational (evidence-based) pharmacotherapy and decreasing health care costs.

It is a further object of the present invention to develop methods and kits for optimizing drug treatment given to patients, particularly those suffering from chronic illnesses and conditions.

It is a further object of the present invention to improve the reliability and predictability of clinical outcomes and/or pharmacotherapy data gathered with respect to new and marketed drugs for submission to regulatory authorities.

In view of the above objects and others, the present invention is directed in part to a method of evaluating the therapeutic response of individual human patients to chronic therapy with a drug, managing health care costs, and optimizing pharmacotherapy comprising a) assembling from a plurality of crossover single patient drug trials a patient population database of information concerning the safety, effectiveness and desirability of a drug administered with a second agent selected from the group consisting of a placebo, a therapeutic alternative for that drug, and a generic equivalent for that drug; b) conducting in a new patient who is a candidate for treatment with the drug a cross-over single patient drug trial of the drug and the same second agent administered to the patient population of step (a); c) comparing the information accumulated from the patient population database with the information from the single patient drug trial of the new patient to aid in the interpretation of the results for the new patient; d) optimizing treatment for the new patient by taking one of the following actions: (i) continuing chronic therapy for the new patient using the same drug and dosage regimen; (ii) changing the dosage regimen of the same drug in order to optimize the dosage regimen for the new patient; (iii) ceasing to treat the new patient with the drug if the new patient is not achieving a desired benefit from treatment, or (iv) changing the new patient to chronic therapy using a therapeutic alternative or generic equivalent of the drug; and e) adding the results from the single patient drug trial of the new patient to the patient population database.

The invention is further directed to a method of evaluating the therapeutic response of individual human patients to chronic therapy with a drug, managing health care costs, and optimizing pharmacotherapy, comprising a) conducting, in a new patient who is a candidate for treatment with a drug, a single patient cross-over drug trial of the drug and a second agent selected from the group consisting of a placebo for that drug, a therapeutic alternative for that drug, a generic equivalent for that drug, and a different dose of the same drug; b) comparing the information accumulated from the single patient drug trial of the new patient with a previously assembled patient population database of information concerning the safety, effectiveness and desirability of the drug administered in a plurality of crossover single patient drug trials with the same second agent administered to the patient population of step (a), to aid in the interpretation of the results for the new patient; and c) optimizing treatment for the new patient by taking one of the following actions: (i) continuing chronic therapy for the new patient using the same drug and dosage regimen; (ii) changing the dosage regimen of the same drug in order to optimize the dosage regimen for the new patient; (iii) ceasing to treat the new patient with said drug if the new patient is not achieving a desired benefit from treatment, or (iv) changing the new patient to chronic therapy using a therapeutic alternative or generic equivalent of the drug; and d) adding the results from the single patient drug trial of the new patient to the patient population database.

The present invention is further directed to a method of predicting the abuse potential of a drug or substance when administered to an individual patient for chronic therapy or used habitually, comprising: a) conducting a single-patient, cross-over drug trial of a drug or substance which is habit forming and a placebo in a new patient who is a candidate for treatment with the drug; b) comparing the information accumulated from a pre-assembled patient population database comprising a plurality of single-patient, crossover drug trials concerning liking scores, abuse potential scores, and patient's desire to re-use the drug administered for chronic therapy and the placebo, with information from the single-patient drug trial of the new patient to aid in the interpretation of the abuse potential and appropriateness of the drug for chronic treatment for the new patient; and c) optimizing treatment for the new patient by taking one of the following actions: (i) continuing chronic drug therapy for the new patient using the same drug and dosage regimen and optionally providing drug counseling; (ii) changing the dosage regimen of the same drug in order to minimize the abuse potential for the new patient and optionally providing drug counseling; or (iii) ceasing to treat the new patient with the drug if the liking scores, the abuse potential scores, and patient's desire to re-use said drug indicate undue abuse potential.

In certain preferred embodiments, this method further comprises adding the results from the liking scores, the abuse potential scores, the desire to re-use the drug from the single-patient drug trial of the new patient and optimization strategy to the patient population database. While the information contained in the patient population pool may be pre-assembled, the method also contemplates the separate assembly of the data for the patient population database as a first step of the method.

The invention is further directed to a method of providing demographic and clinical effectiveness and safety databases obtained from single-patient drug trials comprising a) conducting single-patient, cross-over drug trials of a drug and a placebo in a pool of individual human patients who are candidates for chronic treatment with the drug and obtaining samples of biological materials from the individual human patients before or during their single-patient drug trial; b) identifying genomic and gene expression markers in the pool of individual human patients by testing said biological materials using human DNA microarrays and Single Nucleotide Polymorphism and proteomic and successor technologies and assembling a patient population database of the markers from the pool of individual human patients; c) conducting a single-patient, cross-over drug trial of the drug and the placebo in a new individual human patient who is a candidate for chronic treatment with the drug and obtaining samples of biological materials from the new patient before or during that patient's single-patient drug trial; d) identifying in the new individual human patient genomic and gene expression markers by testing the biological materials using human DNA microarrays and Single Nucleotide Polymorphism and proteomic and successor technologies; e) comparing results from the human DNA and Single Nucleotide Polymorphism and proteomic and successor technologies testing accumulated from the pool of individual human patients with the human DNA and Single Nucleotide Polymorphism and proteomic and successor technologies testing from the new individual human patient to identify correlations between the results from the new individual human patient and the patient population database; and f) optionally (and preferably) adding the results from the single-patient drug trial of the new individual human patient, which results preferably include the optimization strategy chosen, to the results accumulated from the pool. The database is then compared to information collected from the next single-patient trial to guide treatment e.g., by continuing or discontinuing treatment, using an alternative therapy or modifying treatment by using a different dose of the same drug.

In certain preferred embodiments the database can preferably be used for comparing outcomes of previous single-patient trials to statistically predict drug effect. The database can also be used for testing generic equivalents and therapeutic alternative therapies. In certain preferred embodiments, the genomic and gene expression markers comprise surrogate markers of disease etiology and prognosis; drug effectiveness and safety; and lifestyle and intervention synergies. In certain embodiments, the biological material may be, e.g., tissue (e.g., organs, skin, hair, intracellular and extracellular), fluid (e.g., blood, cerebral spinal fluid, amniotic, bone marrow, visceral fluid, gastrointestinal contents, excretory fluid, saliva, mucous and reproductive fluid). The method further contemplates that the information collected from the pool may be pre-assembled for use in such single-patient studies as outlined herein.

The invention is further directed to a method of optimizing clinical outcomes and providing pharmacotherapy in an individual human patient for whom chronic drug therapy is contemplated, comprising: a) determining a first drug for treatment of an individual human patient for whom chronic drug therapy is contemplated, and a second drug which may alternatively be useful for treatment of the individual human patient; b) conducting a single patient cross-over drug trial in the individual human patient via a switchability test utilizing a supply of the first drug; a supply of the second drug, and optionally a supply of placebo; and accumulating information concerning the safety, effectiveness, patient compliance and desirability of the first drug, the second drug and optionally the placebo; c) evaluating whether safety, effectiveness, patient compliance and desirability is acceptable for both the first drug and the second drug; one of the first drug and the second drug; or neither the first drug or the second drug, optionally as compared to the placebo, by comparing the results of the single patient drug trial of the individual human patient with a previously assembled patient population database of information concerning the safety, effectiveness, patient compliance and desirability of the first drug, the second drug and optionally the placebo administered in a plurality of cross-over single patient drug trials, to aid in the interpretation of the results for the new patient; and d) optimizing treatment for the patient by taking one of the following actions: (i) if safety, effectiveness, patient compliance and desirability is acceptable for both the first drug and the second drug, initiating chronic therapy for the individual human patient using the first drug or the second drug, taking into account the relative benefits of each drug based on the results of the evaluation of safety, effectiveness, patient compliance and desirability of the first drug and the second drug as compared to the patient population database, as well as the relative cost of the first drug and the second drug; (ii) if safety, effectiveness, patient compliance and desirability are acceptable for only one of the first drug and the second drug, initiating chronic therapy for the individual human patient using the acceptable one of the first drug or the second drug; (iii) if safety, effectiveness, patient compliance and desirability are not acceptable for either of the first drug and the second drug, discontinuing treatment or repeating steps (b)-(d) utilizing third and fourth alternative drugs, if available. Preferably, the method further comprises adding the results from the single patient drug trial of said individual human patient (which preferably includes the optimization strategy chosen) to said patient population database. While the information contained in the patient population pool may be pre-assembled, the method also contemplates the separate assembly of the data for the patient population database as a first step of the method.

The invention is further related to a method of optimizing clinical outcomes and providing pharmacotherapy in an individual human patient for whom chronic drug therapy is contemplated, comprising: a) determining a drug for treatment of an individual human patient for whom chronic drug therapy is contemplated; b) conducting a single patient cross-over drug trial in the individual human patient via a prescribability test utilizing a supply of the drug and a supply of a placebo; and accumulating information concerning the safety, effectiveness, patient compliance and desirability of the drug, and the placebo; c) evaluating whether safety, effectiveness, patient compliance and desirability is more acceptable for the drug than the placebo; more acceptable for the placebo than the drug; or equivalent for both the drug and the placebo, by comparing the results of the single patient drug trial of the individual human patient with a previously assembled patient population database of information concerning the safety, effectiveness, patient compliance and desirability of the drug and the placebo administered in a plurality of cross-over single patient drug trials, to aid in the interpretation of the results for the new patient; and d) optimizing treatment for the new patient by taking one of the following actions: (i) if safety, effectiveness, patient compliance and desirability are more acceptable for the drug, initiating chronic therapy for the individual human patient using the drug, taking into account the relative benefits of the drug based on the results of the evaluation of safety, effectiveness, patient compliance and desirability of the drug and the placebo as compared to the patient population database; (ii) if safety, effectiveness, patient compliance and desirability are more acceptable for the placebo, initiating chronic therapy for the individual human patient using the placebo or a low risk, less costly alternative therapy; (iii) if safety, effectiveness, patient compliance and desirability are not acceptable for the drug and the placebo, discontinuing treatment or repeating steps (b)-(d) utilizing a second drug and placebo, if available; and thereafter if safety, effectiveness, patient compliance and desirability are more acceptable for the second drug, initiating chronic therapy for the individual human patient using the second drug, taking into account the relative benefits of the second drug based on the results of the evaluation of safety, effectiveness, patient compliance, and desirability of the second drug. The method preferably further comprises adding the results (which preferably includes the optimization strategy chosen) from the single patient drug trial of the individual human patient to the patient population database. While the information contained in the patient population pool may be pre-assembled, the method also contemplates the separate preparation of the data for the patient population database as a first step of the method.

The invention is further directed to a method of optimizing clinical outcomes and providing optimized pharmacotherapy in an individual human patient for whom chronic drug therapy is contemplated, comprising: a) determining a first dose of a drug for treatment of an individual human patient for whom chronic drug therapy is contemplated, and a second dose of the same drug which may alternatively be useful for treatment of the individual human patient; b) conducting a single patient cross-over drug trial in the individual human patient via a dosability test utilizing a supply of the first dose of drug and a supply of the second dose of the same drug; and accumulating information concerning the safety, effectiveness, patient compliance and desirability of the first dose of drug, and the second dose of the same drug; c) evaluating whether safety, effectiveness, patient compliance and desirability are more acceptable for the first dose of drug than the second dose of the same drug; the second dose of drug than the first dose of the same drug; or neither the first dose of drug or the second dose of the same drug, by comparing the results of the single patient drug trial of the individual human patient with a previously assembled patient population database of information concerning the safety, effectiveness, patient compliance and desirability of the first dose of drug and the second dose of the same drug administered in a plurality of crossover single patient drug trials, to aid in the interpretation of the results for the new patient; and d) optimizing treatment for the new patient by taking one of the following actions: (i) if safety, effectiveness, patient compliance and desirability is more acceptable for the first dose of drug, initiating chronic therapy for the individual human patient using the first dose of drug, taking into account the relative benefits of each dose of drug based on the results of the evaluation of safety, effectiveness, patient compliance and desirability of said first dose of drug and said second dose of said same drug as compared to the patient population database, as well as the relative cost of the first dose of drug and the second dose of the same drug; (ii) if safety, effectiveness, patient compliance and desirability are more acceptable for the second dose of drug than the first dose of the same drug, initiating chronic therapy for the individual human patient using the second dose of drug; (iii) if safety, effectiveness, patient compliance and desirability are not more acceptable for either of the first dose of drug and the second dose of the same drug, discontinuing treatment or repeating steps (b)-(d) utilizing new first and second dose of the same drug or a first and a second dose of a second alternative drug, if available; and, thereafter, if safety, effectiveness, patient compliance and desirability are more acceptable for either the new or the first or second dose of the alternative drug, initiating chronic therapy for the individual human patient using that dose of the drug or second alterative drug. The method preferably further comprises adding the results from the single patient drug trial of the individual human patient (which preferably includes the optimization strategy chosen) to the patient population database. While the information contained in the patient population pool may be pre-assembled, the method also contemplates the separate preparation of the data for the patient population database as a first step of the method.

Thus, in one aspect, the invention includes a method of treating human and veterinary illnesses. The method includes:

a) providing to a pool of humans or animals in need of such treatment a test kit containing:

i) a supply of a drug indicated or proposed for the treatment of an illness;

ii) a supply of a placebo substantially identical in appearance and presentation to the drug;

iii) a questionnaire designed to elicit from each pool member to be treated information concerning the actual usage, safety, effectiveness and desirability of the selected treatment;

b) administering the drug and placebo to each member of the pool according to a random, double-blind schedule;

c) assembling a database from the pool based on the answers provided from the individual questionnaire;

d) revealing the random schedule and comparing the data obtained from known drug and placebo treatment periods;

e) providing a test kit containing the same materials as set forth in a) to a human or animal also in need of such treatment to obtain a separate or second set of data concerning the safety, effectiveness and desirability of said treatment;

f) administering the drug and placebo to the human or animal according to a random, double-blind schedule;

g) assembling the second or separate database based on the answers provided to the questionnaire;

h) revealing the randomized schedule to uncover the drug and placebo treatment periods;

i) comparing the data obtained from the pool with that obtained from the single human or animal trial to determine a treatment which provides optimal therapeutic effect and quality of life of the human or animal with the drug; and

j) administering to the human or animal a dosing regimen consistent with the optimal regimen.

The new dosing regimen for optimal therapeutic effect and quality of life can also be retested, if and when deemed appropriate, by the clinician and/or patient.

The method is suitable for evaluating and validating any prescription or non-prescription treatment regimen or medication for individuals as well as demographic groups. Using this method, one can periodically obtain further outcome information on tested individuals.

Other aspects of the invention include a method and kit for determining therapeutic alternatives and verifying generic equivalence of known medications. The method includes:

a) providing to a human or animal a test kit containing:

i) a supply of a drug indicated for the treatment of an illness;

ii) a supply of a therapeutic alternative, a generic equivalent candidate or a different dose of the drug substantially identical in appearance to the drug;

iii) a questionnaire designed to elicit from the human or animal information concerning the safety, effectiveness and desirability of the treatment for the human or animal;

b) administering the drug and therapeutic alternative to the human or an animal according to a randomized, double-blind schedule;

c) assembling a database by eliciting from the human or animal answers to the questionnaire; and

d) revealing the random arrangement schedule to determine the relative effectiveness of the therapeutic alternatives in the human or animal by comparing the data obtained from knowing drug and alternative treatment periods.

In certain preferred embodiments of each of the forgoing methods, the plurality of single patient drug trials which makes up the patient population database in each of the embodiments of the invention are conducted according to a randomized, double blind schedule.

In certain preferred embodiments of each of the forgoing methods, the single patient drug trial for the new patient who is a candidate for treatment with the drug in each of the embodiments of the invention is conducted in randomized, double-blind, cross-over fashion.

In certain embodiments of each of the forgoing methods, the single-patient clinical trial for the new individual human patient (and for the single patients whose data is assembled into the patient population database) is conducted in parallel fashion. Also, for single-patient clinical trials conducted in parallel fashion, the trials may be conducted in open-label, single-blind or double-blind fashion.

In certain embodiments of each of the foregoing methods, the patient population database is stored on a computer, and is in certain further embodiments accessible from a remote location.

In certain embodiments for the forgoing methods, herbal or dietary preparations or so-called “complimentary medicines” may be used in place of a drug which has been approved by regulatory agencies for indicated diseases or conditions.

In certain preferred embodiments, the method further comprises assembly of a patient population database by providing to each patient in the patient population a test kit containing a supply of the drug; a supply of placebo; and a questionnaire designed to elicit from the patient population information concerning the actual usage, safety, effectiveness and desirability of the drug. In certain further preferred embodiments, the method further comprises assembly of the information from the individual patient drug trial by providing to the individual patient a test kit containing a supply of the drug; a supply of said placebo; and a questionnaire designed to elicit from the patient or caretaker information concerning the actual usage, safety, effectiveness and desirability of the drug.

In certain preferred embodiments of each of the foregoing methods, the data obtained from the single-patient trial of the individual patient and from the patient population database is preferably further assembled from objective testing methodologies collected before and during the single-patient drug trial. The objective testing methodologies utilized by the present invention include, but are not limited to, the monitoring of blood pressure, cholesterol, blood sugar, glycosylated hemoglobin and combinations of any of the foregoing. Monitoring of the objective data may be performed, e.g., by the individual patient during the single-patient drug trial or by the physician or caretaker.

In other preferred embodiments, the data obtained from the single-patient trial of the individual patient and from the patient population database concerning the method for predicting the abuse potential of a drug or substance, is preferably further assembled from certain specific objective testing methodologies which include, but are not limited to, mood (measured by a visual analog scale (VAS), sedation (measured by VAS), Respiratory rate (breaths per minute), Pupil size (measured by pupillometry) and any combinations of the foregoing.

The invention is further directed to the use of test kits (e.g., as described herein) containing a supply of drug; a supply of a second agent, such as placebo, a therapeutic alternative, a generic alternative, or a different dose of the drug; and a questionnaire designed to elicit from the patient population information concerning the actual usage, safety, effectiveness and desirability of the drug, in any of the foregoing methods for evaluating the response of individual human patients to chronic therapy with a drug.

The invention is further directed to targeting additional appropriate alternative drugs and timings for single-patient trials including drug holidays and re-tests.

In yet additional embodiments of the foregoing methods the results of the optimization strategy are preferably added (e.g., input from a remote location by tying into a central database on-line computer) into the patient population database. The methods also preferably include the pre-assembly of the patient population database to be used in future single-patient tests as contemplated herein.

There are many advantages associated with the present invention. For example, patients benefit by the assurance of treatment with appropriate drug and dosing regimens. The method is particularly useful before committing a patient to a long term drug treatment regimen. Documented evidence of the benefit is provided. Unnecessary side effects and expense can be avoided. Government agencies could also benefit by the availability of a dynamic database on drug efficacy and safety in individuals.

The inventive method also provides an alternative means of approving new drugs. In this aspect, the new drug or therapeutic alternative could be tested according to the methods described herein against a placebo or a known effective agent and/or indicated therapy in individuals and/or a pool of suitable candidates. This is a particular advantage to the pharmaceutical industry and affords a method to validate the therapeutic equivalence of generic drugs as well as non-generic therapeutic alternatives.

Insurers and managed care organizations could also benefit by having a reliable “second opinion” to help avoid expensive, prolonged, unneeded, or toxic treatments, and promote utilization of safe and effective therapies.

The present invention provides advances over prior art single-patient drug trials (n=1) by optimizing treatment for the individual. Unlike (n=1) studies, which by definition, had a sample size of one, the invention includes comparing the data obtained from the individual with a database accumulated for an entire tested population, referred to as a pool herein. This results in the opportunity to create a prospective, frequently updated epidemiological database which has value not only for regulatory approvals or post-marketing surveillance of drug safety and efficacy, but also for optimizing outcome in individual as well.

Managed care has historically managed drug formulary inclusion/exclusion decisions based, for the most part, on population efficacy and safety statistics weighed against financial costs. The present invention can significantly improve upon these decision tools by providing for an evidence-based, individual patient formulary management control system which can be used in conjunction with group generalizations created by single patient drug trials and existing pharmacoeconomic data. An important aspect of the invention is that, pursuant to the method, formulary and individual patient decisions are made based on individual patient efficacy and safety outcomes, rather than cost, as found in most formulary systems, particularly those which encourage therapeutic decisions using step-care methodologies.

The present invention is useful because it can significantly reduce cost to the health care system without any compromise to patient health and well being. In fact, the savings can be enjoyed concurrently with improved patient outcomes due to refined use of individual therapeutic outcome data.

Another object of the present invention to provide a method of gaining FDA approval and surveillance post-approval for new drugs which have been discovered for the treatment of chronic illnesses and conditions.

### BRIEF DESCRIPTION OF THE DRAWINGS

The following drawing is illustrative of embodiments of the invention and is not meant to limit the scope of the invention as encompassed by the claims.

FIG. 1 shows a step-by-step analysis of the single-patient clinical trial flowchart used for evaluating appropriateness of specific drug treatments.

### DETAILED DESCRIPTION

The method of invention takes over where group clinical trials and the FDA review for safety and effectiveness end. It provides the practicing physician with an objective, scientifically valid tool for determining how best to treat a given patient by providing patient-specific data that are not obtainable from group clinical trails.

The present invention includes a method and kit for determining the appropriate treatment for a chronic illness or condition. The method includes: (a) providing to a pool of humans or animals in need of such treatment a test kit containing: (i) a supply of a drug indicated or proposed for the treatment of an illness or condition; (ii) a supply of a placebo substantially identical in appearance to the drug; (iii) a questionnaire designed to elicit from each member of the pool information concerning the safety, effectiveness and desirability of the selected treatment; (b) administering the drug and placebo to each member of the pool according to e.g., a random, open label, single blind or double-blind schedule; (c) assembling a database by eliciting from the pool data from the answers to the questionnaire; (d) revealing the random schedule to uncover drug and placebo treatment periods; (e) providing a test kit containing the same materials as set forth in a) to a new human or animal patient also in need of such treatment to obtain a second or separate set of data concerning the safety, effectiveness and desirability of the treatment; (f) administering the drug and placebo to the human or animal according to, e.g., a random, double-blind schedule; (g) assembling a second or separate database by eliciting from the human or animal caretaker answers to the questionnaire; (h) revealing the random schedule and comparing the data obtained as a result of known active(s) or placebo(s) treatment periods; (i) comparing the results obtained from the first set of data obtained from the pool with the second set of data obtained from the single trial to determine an optimal treatment for the human or animal with the drug; and () administering to the human or animal a treatment consistent with the optimal treatment, based upon individual and group outcome.

Results from the individual, and post-study follow-up data can also be added to the general database.

Even after the data from the questionnaires is obtained, the caregiver can continue to periodically use the same kit or other kits with different test articles, analyzing the further results for relative scoring, or monitoring further treatment based on physician and patient awareness of study results.

For purposes of description of the present invention, certain terms are described below. Generally, however, the terms have the commonly understood meaning known to those of ordinary skill in the art.

Drug shall mean a medicament, biologically active ingredient, or pharmaceutical dosage form containing an active ingredient effective for one or more medical conditions. The drug may be in any known dosage form including tablets, capsules, solutions, elixirs, ointments creams, etc. For purposes of this invention, drug may be an herbal, so called “complementary medicine,” alternative medicine, dietary supplement or other product that has not been approved as a drug by a regulatory agency.

Placebo shall mean an inert or inactive dosage form having an appearance and/or other organoleptic/sensory characteristics totally, substantially or virtually identical to an active drug.

Treatment shall mean administering a customary amount of a drug or a placebo for the purpose of alleviating or curing a disease, condition or deficiency.

Optimal or optimizing treatment means a treatment regimen which has been adjusted or validated in view of a comparison of objective data relating to one or more treatment periods with one or more active medicament(s) and one or more of placebo, a therapeutic alterative or a generic equivalent. This is further adjusted by consideration of outcomes from similarly tested populations. Treatments consistent with optimal treatment are those which adjust the time, manner or amount of a drug or therapy for maximum effect, or even cease to treat with the drug or therapy or use as a therapeutic alternative.

Supply means a quantity sufficient for completing a statistically valid evaluation of a treatment method in an individual.

Therapeutic alternative (therapeutic substitute) means a medicament having a non-identical chemical composition from a known medicament but achieves substantially the same bioeffect in an individual. For example, in the wake of recent controversies involving the abuse of ephedrine or phenylpropanolamine (PPA; commonly used in diet preparations or in nasal decongestant formulations), recommendations have been issued for the pharmaceutical industry to use the more safe therapeutic alternative, pseudoephedrine. For purposes of this invention an alternative therapy may also be a different dose of the particular drug.

A generic drug or medicament (“generic equivalent”) means a substantially identical active ingredient to a known composition, or a copy of an originally approved drug that is bioequivalent to the originally approved (brand name) drug. Bioequivalent refers to the rate and extent to which the active agent is absorbed from the dosage formulation and the extent it becomes available at the site of action. A generic drug is a drug that does not show any significant difference when administered at the same dosage ratios of the brand name drug under experimental conditions, either as a single or multiple dose. The Food and Drug Administration (FDA) publishes a listing of generic equivalent drug products entitled, Therapeutic Equivalence Evaluations (“Orange Book”). An example of a typical generic equivalent is diazepam, which is the generic equivalent of the brand name Valium®.

Chronic shall mean treatment of a condition which lasts an indefinite period of time. Treatments amounting to more than a single course of therapy. Maintenance dosing regiments are also contemplated.

Prolonged therapy shall mean therapy wherein doses are administered to a patient over a period of greater than 10 days, including multiple episodes or recurrences of shorter duration. For example, a psoriasis or herpes episode may require intermittent treatments of less than 10 days but the condition requires prolonged therapy.

The term “open-label” shall be consistent with its known meaning and includes known techniques such as single or multiple crossover techniques well known to those of ordinary skill.

Open-label means that the patient or caretaker if appropriate, and care-giver know exactly when the drug, alternative drug or placebo is given.

The term “single-blind” shall be consistent with its known meaning and includes known techniques such as single or multiple crossover techniques well known to those of ordinary skill. Single-blind means that the patient or caretaker if appropriate, do not know exactly when the drug, alternative drug or placebo is given, but the care-giver does know exactly when the drug, alternative drug or placebo is given.

The term “double-blind” shall be consistent with its known meaning and includes known techniques such as single or multiple crossover techniques well known to those of ordinary skill. Double-blind means that the patient or caretaker if appropriate, and care-giver do not know exactly when the drug, alternative drug or placebo is given.

The term “parallel” shall be consistent with its known meaning and includes know techniques to randomize one of two or more treatment groups to usually receive the assigned treatment during the entire trial. The treatments assigned to the two groups differ. Each group generally receives either trial medicine or placebo, one of two different trial medicines, or one of two doses of the same trial medicine. Two placebo medications could also be evaluated. One variation of the parallel design is for each group to receive alternating (and escalating) doses of the same drug.

The term “switchability test kit” means a test kit that is made up of a drug (Drug A) and another drug (Drug B) and optionally placebo which is used for comparing the effectiveness and safety of Drug A, Drug B, and optionally placebo.

The term “prescribability test kit” means a test kit made up of an active drug and placebo which is used for comparing the superiority of active drug versus placebo and visa versa.

The term “dosability test kit” means a test kit made up of a low dose of a drug and a high dose of a drug which is used for comparing the overall safety, effectiveness and desirability of the lower dose versus the higher dose of drug.

The term “overall profile” means the usage, safety, effectiveness, and desirability of treatment data of a drug which is obtained based on the answers to the questionnaires or objective measures.

For purposes of description, the method and kit can be described as a Single-Patient Assessment System (SPAS). The SPAS provides a health care practitioner with objective data based on each individual patient's unique circumstances, allowing therapy to be tailored to individuals needs. In addition, the unique method generates prospective, directly measured epidemiologic safety and effectiveness data. Pharmaceutical companies can use this data to gain regulatory approval for new indications or to differentiate effectiveness and/or safety benefits between competing products, and to provide pharmaceutical manufacturers, government and health care organizations with demographic and usage data on products. Importantly, the database can also be used by government agencies to monitor the safety and effectiveness of drugs in the marketplace. Using statistical sub-group analyses, data can be generated to define the level of effectiveness or safety in various special populations. For example, data can be segregated by age, disease severity, onset of illness, and concurrent medications.

Further, it should be noted that while the present invention is for a method wherein treatment is optimized, the actual treatment given to a patient (human or animal) will be determined by a physician, veterinarian, or other healthcare professional. The step of optimizing treatment can only be made by a healthcare professional with the legal right to prescribe drugs. Nothing contained in this application shall be construed so far as to interfere with a physician/patient relationship or imply that an individual other than a physician or legally authorized professional shall be dispensing medical diagnosis, treatment, or other services considered the practice of medicine pursuant to state laws.

One preferred embodiment to the invention includes the use of SPAS to demonstrate the effectiveness of the specific treatment for the specific individual, that is, to document the probability that the medication is beneficial without causing unacceptable side effects. Specifically, the system consists of a clinical evaluation kit which generates definitive guidance regarding the safety and effectiveness of drug therapy in each individual patient. The kit contains a full supply of medication to be evaluated and/or placebo, as well as all instructions and evaluation instruments for professionals and patients.

A preferred feature of the present invention is the double-blind manner in which the drug or placebo, or alternative drug, is being administered. Both the patient and the physician are unaware of what dose is given. This is advantageous since placebo and active drug are randomly administered and look identical to eliminate any bias in the results. A neutral observer/administrator keeps the record of the randomized arrangement, assembles the data from completed questionnaires and after completion of the test, “breaks the code” to reveal the schedule of drug and/or placebo doses and analyzes the accumulated data. The physician and/or patient is/are then given a report on the usage, effectiveness, safety and desirability of the drug treatment in question. The report has the feature of being validated because the data obtained, at least in part, from the single patient is compared to data obtained from a pool of individuals who also required treatment, were assigned a test kit, and were followed-up for usage, effectiveness, safety and desirability data post-testing. This can be used for guidance in directing further therapy, referred to herein as a treatment consistent with optimal treatment. The results of individual assessments can be monitored, with subsequent outcomes added to the database. Data generated from a pool of individual studies can then form the basis of a large population database reporting system which serves to further validate the effectiveness of any singular trial or single indication for a medication.

The SPAS includes means for providing the drug(s), placebo(s) and questionnaire(s) such as a kit. The kit may contain convenient cards which contain a sufficient supply of active drug(s) and placebo(s), or therapeutic alternative(s) or generic equivalent(s) in blister packages labeled with the time of dosing. For example, a kit may contain eight cards for a required trial, each card corresponding to one of eight weeks of treatment, and each kit may contain daily regimens of either active drug or placebo, at carefully selected times during the eight week period. The tablets in the card are often “blinded” so that neither the patient nor the physician is aware of which preparation is received at any given time. In an emergency, the random arrangement can be broken. Under normal circumstances the code will not be made available to the patient or physician/care taker, thereby eliminating any bias in the results.

When the SPAS of the present invention comprises a depression test kit for optimizing chronic treatment of a drug for the treatment of a psychiatric condition, in addition to the questionnaire used to determine safety, effectiveness and desirability of the test drug, the depression test kit preferably utilizes a depression test scale for evaluating an individual patient's depression symptoms throughout the course of the single-patient drug trial. The depression test scale utilized in the SPAS is a Beck Depression Fast Screen (BDI-Fast Screen) developed by The Psychological Corporation, Harcourt, Brace and Company, San Antonio, Tex. The BDI-Fast Screen consists of groups of statements designed to elicit from the individual answers regarding the individual's depression symptoms, e.g., I do not feel sad, I feel sad much of the time, I am sad all of the time, I am so sad or unhappy that I can't stand it. The depression scale questionnaire is filled out at the completion of an appropriate cycle, such as a seven-day cycle, immediately after the individual has completed taking the medication contained in the envelope. These depression scale questionnaires will be submitted along with the safety, effectiveness and desirability questionnaires throughout the course of the single-patient drug trial. One skilled in the art will appreciate that other depression test kits may be utilized instead of the Beck Depression Fast Screen, such as Beck Depression Inventory II (BDI-II), or the psychiatric rating scales set forth in Marder S R, “Comprehensive Textbook of Psychiatry/VI 6^{th} ed., Baltimore, Md.: Williams & Wilkins; 1995;1:619-635. These alternatives are for illustration purposes only, and are not meant to limit the scope of the invention.

At various times during the evaluation, the program prompts the patient, physician and/or guardian/observer to fill out questionnaires or the instruments which assess numerous usage, effectiveness, safety and desirability of treatment variables relating to improvements in physical and behavioral symptoms.

At the end of the study, all drug cards (used and unused) as well as uncollected questionnaires and objective data are returned and the results are evaluated. These results are provided to the physician, caregiver and patient so that guidance can be provided regarding the usage, safety, effectiveness and desirability of the treatment for the tested patient. These data can also be added to a master database along with other data on family history, demographics, socioeconomic factors, and post-study outcome.

The questionnaires may be transmitted and answered by electronic media such as telephone, facsimile and the internet.

Numerous drugs and indications can be evaluated using the methods of the present invention. Suitable illnesses and conditions for which the present invention can be used include, without limitation, asthma, cancer, epilepsy, schizophrenia, minimal brain dysfunction, mania, depression, anxiety, alzheimer's disease, attention deficit disorder (ADD), hypertension, angina, congestive heart failure cardiac arrhythmias, pain, metabolic and endocrine disorders, obesity (e.g. treatments for weight reduction), neurologic diseases, immunologic diseases, eye and ear disorders, dental diseases, and sleep disorders.

Suitable drugs for evaluation include, without limitation, those agents currently approved for the above-identified conditions as well as agents waiting approval and new chemical entities. For example, the drug can be selected from a drug for treating hyperkinetic behavior, anti-asthmatic agent, dental agents, anti-epileptic agents, anti-psychotic agents, anti-depressants, cardiovascular agents, respiratory agents, neurological agents, antihypertensive agents, diabetic agents, steroidal and non-steroidal anti-inflammatory agents, opiates, narcotic and non-narcotic analgesics, hematologic agents, musculoskeletal agents, anti-anxiety agents, gastro-intestinal agents, dermatologic agents; and anti-allergy medications. Other categories not specifically mentioned are intended as well. Particular agents well suited for the methods of the present invention included methylphenidate, steroids, such as androgen and estrogen-containing agents, anti-asthmatic agents, cardioactive agents, and antidepressant agents.

Additional agents include those used for the treatment of oral, mucous membrane, nasal, surgical, musculoskeletal, central nervous system, urinary tract, psychiatric, renal, neurologic, genital disorders (e.g., erectile dysfunction), genito-urinary, podiatric, chiropractic, and geriatric conditions, as well as agents used for treatments such as acupuncture, allopathy, homeopathy and osteopathy can also be evaluated.

It is to be understood that where veterinary treatments and therapies are to be tested, the questionnaires and assembly of data are provided by human caretaker/observers. Furthermore, it is to be understood that the term questionnaire refers generally to a means by which information can be related back to the evaluator. The results need not be transmitted in written form. Computer-assisted and telephone assisted data recording and communication devices and measuring instruments can also be part of the database assembly step.

An additional list of uses includes:

1) Socially/medically controversial uses for drugs where the relationship of risk to benefit is not well defined. For example, depression, asthma, ADD and hyperkinetic behavior are representative chronic ailments which can be evaluated and available treatments can be challenged.

2) Chronic disease states which may or may not benefit from long term drug treatment. Controlled drug “holidays” are needed to test if chronic medication is paradoxically compromising quality-of-life, has no effect or is helping and should be continued. Category examples include cardiovascular disease, hypertension, and arthritis.

3) “Compassionate” Investigational New Drug Application (IND) trials for drugs/indications which command a fast track regulatory approval process, such as drugs used for treatment of AIDS. Pharmaceutical companies can pursue early “compassionate” marketing in the form of a drug trial in subjects who urgently need the new therapy and the aggregated database can be submitted for regulatory approval as pivotal trials. Also, early New Drug Application (NDA) approval can be pursued by carefully controlling drug use, investigational documentation and data analysis in the community-practice setting. These regulatory strategies can be economically and effectively accomplished using Single-Patent Assessment Systems (SPAS).

4) Clinical comparison between innovator and generic drugs. Single-Patient Assessment Systems (SPAS) can be used to validate or invalidate use of generic drugs for regulatory or marketing purposes. Single-Patient Assessment Systems (SPAS) can be used to gain approval for generic drugs which are not readily approved by traditional bioequivalence testing. The method and kit can offer a consumer assurance of a successful switch from the innovator's product, and assurance that the drug actually improves his or her quality-of-life.

Another example of the method and kit is for evaluating new or generic drugs, evaluating new indications for marketed drugs or therapeutic equivalents. This includes determining a therapeutic alternative of known medications for an individual or animal requiring treatment.

This aspect includes:

a) providing to a human or animal a test kit containing:

i) a supply of a drug indicated for the treatment of an illness;

ii) a supply of a therapeutic alternative substantially identical in appearance to the drug;

iii) a questionnaire designed to elicit from the person or animal caretaker information concerning the usage, safety, effectiveness and desirability of the selected treatment;

b) administering the drug and therapeutic alternative to the person or animal according to an open-label, or random, single-blind or double-blind schedule;

c) assembling a database from the answers to the questionnaires; and when a single-blind or double-blind schedule is utilized;

d) revealing the random arrangement schedule to determine the effectiveness of the therapeutic alternative by comparing the results obtained from known drug and alternative treatment periods.

This method may also included additional steps which serve to validate the data obtained in any single trial. The steps are:

e) providing the same type of test kit to a pool of humans or animals in need of such treatment and obtaining from the pool a second set of data including post-study follow-up information where appropriate, concerning the safety, effectiveness and desirability of treatment with the drug and therapeutic alternative; and

f) comparing the data obtained from an individual with that obtained from the pool to verify the effectiveness of the therapeutic alternative.

The method described herein also contemplates that the therapeutic alternative is a generic equivalent for the drug and/or the same drug but at a different dosage or even the same dosage.

The present invention has a myriad of other uses. For example, it can be used to test, confirm or verify a particular therapy's safety and effectiveness. It can also provide demographic, marketing, sales or professional usage information. New indications, patterns of use, compliance, therapy relationships to other disease states, relationships between concomitant medications, and laboratory result relationships can be uncovered. The present invention can be used in regulatory filings, dose titration, open-label, single-blind, double-blind, placebo controlled, crossover, parallel, food effect, dose proportionality, bioavailability single dose, multiple dose and market research studies. Age effects, socioeconomic effects, sex effects, and disease effects can also be determined. Moreover, the role of heredity, diet, geographic location, demographic, occupation, epidemiology, patient education, drug interactions, dose response, time to onset, dosage individualization, regimen individualization, dose finding, dose ranging, rising dose, dose titration or overdose can be determined.

The kits of the present invention also have value to physicians. Legal documentation concerning rational drug therapy, compliance, monitoring, documentation of decision making, appropriateness of therapy, ease of following instructions for administration of therapy and documentation of safety and effectiveness are all achieved by the inventive process. The method gives a reason for patient compliance and drug effects, a mechanism for follow-up of therapy, the ability to ease concerns about safety and effectiveness. The ability to use blinded placebo treatment methods and the ability to remove bias from decision making, ease of screening out psychosomatic illnesses are also provided. The kit can provide drug holidays in blinded manner to foster compliance, make available objective feedback and an unbiased and rational approach to therapy. The kit allows the involvement of all physicians and/or patients in clinical trials, not only academia and clinical research organizations, early patient participation in therapy, decreases time for regulatory submissions with less initial use of specialists in clinical trials and less dependence on traditional clinical investigators. All of these features decrease overall medical costs, decrease the costs of new drug development, increases accuracy of diagnoses and potentially decreases malpractice.

The kit and method has value to patients by lessening the fear of inappropriate medicine and providing the feeling that something important is being done. Individualization of therapy for the patient, decreased side effects, increased effectiveness, decreased risk of treatment, controlled drug holidays are all realized. Patients have the enhanced ability to use new and unapproved treatments when needed with the enhanced ability to participate in clinical trials. The kit decreases overall costs of treatment, eliminates unnecessary therapies and tests, reminds patients when to administer the drugs, prevents under or overdoses, fosters relationships with clinicians, and increases understanding of disease and drug.

Industry will benefit from the invention by having a means to gain drug approval, a marketing tool; a reduction of clinical trial costs, better clinical trials, larger clinical trial databases, broader patient populations for clinical trials, the ability to conduct well controlled, small scale, initial clinical trials; a means for post-marketing surveillance, as well as a means to document therapeutic bioequivalence.

The kit and method's value to government is realized by providing a means to remove clinician/company/patient bias in important therapy; protecting the public from inappropriate drug use, decreasing the cost of public health, and lowering the cost of effective clinical assessment of new and existing drugs, more rapidly approving new drugs and indications, providing highly controlled methods to deploy needed but unapproved treatments; and providing new methods for phase I through phase IV treatment evaluations.

Third party healthcare organizations, insurers and managed care organizations benefit by the assurance of need for expensive and/or potentially dangerous therapies, documented need or lack of need for therapies, overall decreased cost of treatment, decreased use of unneeded and/or multiple therapies, improved clinical outcomes, decreased iatrogenic disease, scientifically-driven drug formulary systems.

Pharmacists benefit by the availability of new products, enhanced role in patient care, greater interaction with patients and with other health care professionals.

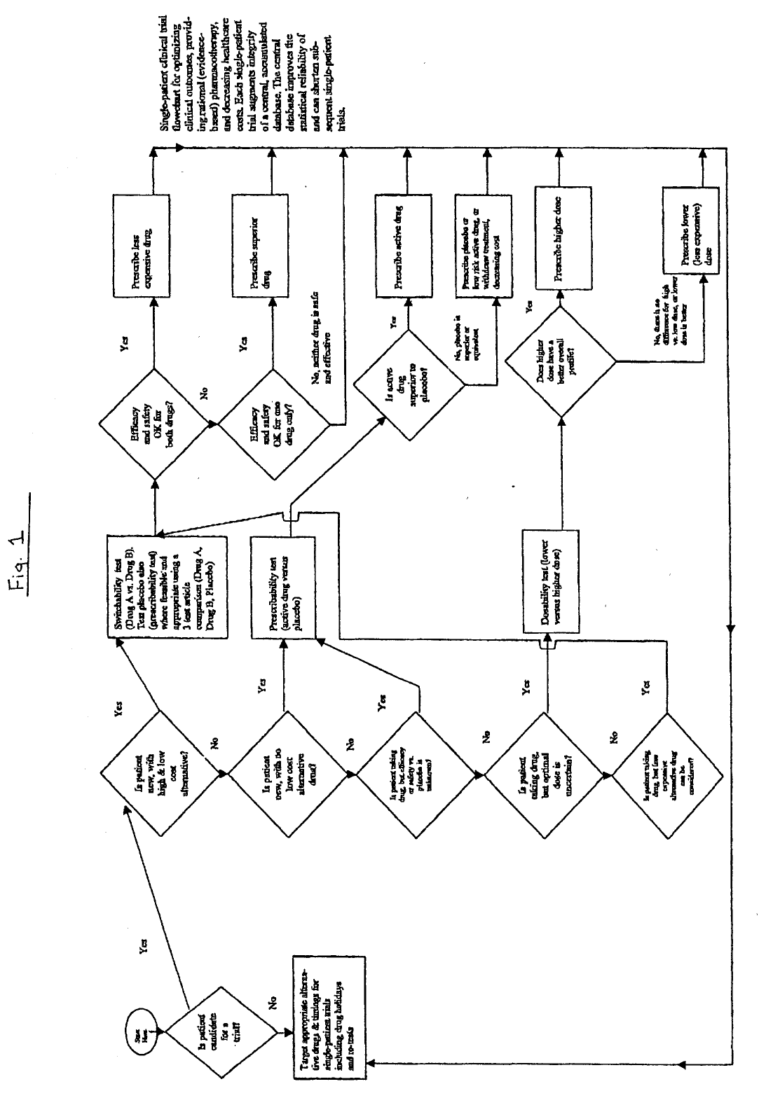

An example of the method of providing demographic and clinical efficacy and safety databases obtained from single-patient drug trials may be further understood by the flowchart which is provided in FIG. 1, and is explained as follows:

Step 1—the patient is evaluated to determine if he/she is a candidate for the trial.

Step 2—comprises a five-prong test to determine which one of three test kits the patient will be assigned. In the first prong the physician evaluates whether the patient is new, with high and low cost drug alternatives. If the answer is “yes”, the patient is assigned to the Switchability test kit in Step 3. If the answer is “no”, the physician considers the second prong analysis which evaluates whether the patient is new with no low cost alternative drug. If the answer is “yes”, the patient is assigned the Prescribability test kit in Step 3. If the answer is “no”, the physician considers the third prong analysis which evaluates whether the patient is already taking a drug, but the effectiveness or safety vs. placebo is unknown. If the answer is “yes”, the patient is assigned the Prescribability test kit. If the answer is “no”, the physician considers the fourth prong analysis which evaluates whether the patient is taking drug, but the optimal dose is uncertain. If the answer is “yes”, the patient is assigned the dosability test kit in Step 3. If the answer is “no”, the physician considers the fifth prong analysis which evaluates whether the patient is taking drug, but less expensive alternative drugs can be considered. If the answer is “yes”, the patient is assigned the switchability test kit. If the answer is “no”, the physician then targets appropriate alternative drugs and timings for single-patient trials including drug holidays and re-tests.

Step 3—comprises the patient receiving one of the three above-mentioned test kits which he/she was assigned (the switchability test kit, the prescribability test kit, and the dosability test kit). The switchability test kit compares Drug A vs. Drug B and, where feasible and appropriate, the switchability test kit compares Drug A vs. Drug B vs. Placebo using a three test article. The prescribability test kit compares active drug vs. placebo and the dosability test kit compares lower vs. higher doses of the active drug.

Step 4—comprises a four-prong test. The first and second prongs analyze the effectiveness and safety for patients assigned to receive the switchability test kit; the third prong analyzes the superiority of active drug vs. placebo in patients assigned to receive the prescribability test kit; and the fourth prong analyzes the overall profile of the active drug(s) and placebo in patients assigned to receive the dosability test kit.

A patient assigned to receive the switchability test kit is evaluated under the first prong to determine whether the effectiveness and safety is acceptable for both drugs. If the answer is “yes”, the patient is prescribed the less expensive drug. If the answer is “no”, the patient is evaluated under the second prong to determine whether the effectiveness and safety is acceptable for only one of the drugs. If the answer is “yes”, the patient is prescribed the superior drug. If the answer is “no”, safety and effectiveness are not acceptable for either drug, then the physician targets appropriate alternative drugs and timings for single-patient trials including drug holidays and re-tests.

A patient assigned to receive the prescribability test kit is evaluated under the third prong to determine whether the active drug is more acceptable to placebo. If the answer is “yes”, the patient is prescribed the active drug. If the answer is “no”, placebo is more acceptable or equivalent, the patient is prescribed placebo or low risk active drug to decrease the cost.

A patient assigned to receive the dosability test kit is evaluated under the fourth prong to determine whether a first (higher) dose of drug is more acceptable than a second (lower) dose of the same drug. If the answer is “yes”, then the patient is prescribed the first (higher) dose. If the answer is “no”, neither dose is more acceptable than the other, then the patient is prescribed the lower (less expensive) dose.

Step 5—combines the analysis of the previous four steps. When treatment is indicated the patient is assigned to one of six treatment alternatives. The six treatment alternatives are as follows:

| | | | |
|---|---|---|---|
| 1. | Prescribe less expensive drug. | 5. | Prescribe higher dose. |
| 2. | Prescribe more acceptable drug. | 6. | Prescribe lower (less |
| 3. | Prescribe active drug | | expensive) dose. |
| 4. | Prescribe placebo or low risk active | | |
| | drug, decreasing cost. | | |

Once a patient has been assigned to one of the six treatment alternatives in Step 5, it is preferable for them to be evaluated throughout the treatment period. If it is determined that the treatment assigned becomes ineffective (inefficacious, unsafe) then the analysis proceeds to Step 6.

Step 6—comprises targeting alternative drugs and timings for additional single-patient trials which include drug holidays and re-tests.

Even after the data from the questionnaires is obtained, the care giver can continue to periodically use the same kit or other kits with different test articles, analyzing the further results for relative scoring, or monitoring further treatment based on physician and patient awareness of study results.

The present invention preferably includes the use of the SPAS to demonstrate the effectiveness of the specific treatment for the specific individual, that is, to document the probability that the medication is beneficial without causing unacceptable side effects. Specifically, the system consists of a clinical evaluation kit which generates definitive guidance regarding the safety and effectiveness of drug therapy in each individual patient. The kit contains a full supply of medication to be evaluated and/or placebo, as well as all instructions and evaluation instruments for professionals, patients and, if appropriate, caretakers.

Pharmacogenomics and proteomic approaches are known in the art, for example, as discussed in U.S. Pat. No. 6,180,358. These approaches are described as providing the means to identify genes, gene expressions and proteins that predict drug response (known as “a genome-wide association”) and rely primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Messenger RNA and proteomic markers may also be similarly involved in a disease process. Given a genetic map based on the occurrence of such SNPs, individuals can be grouped into genetic categories depending on a particular pattern of markers in their individual genome. Theoretically, treatment regimens can be tailored to groups of genetically similar individuals, taking into account traits that may be common among such genetically similar individuals.

Other than Opt-e-scrip/Opt-e-pop (the trademarks for single-patient drug trials (Opt-e-scrip™) used in conjunction with the accumulated database (Opt-e-pop™)) from such clinical trials method, there is currently no other economically viable way for genomics and proteomic companies to gain access to large populations of ill individuals. This is because there is no other way for genomics and proteomic companies to access large prospective crossover clinical trial data in actual clinical settings without incurring massive clinical trial development costs.

The Opt-e-scrip™ trial preferably consists of a definitive, single-patient, double-blind, multi-crossover clinical trial measuring drug safety and effectiveness for a test drug vs. either placebo, a therapeutically similar drug, or a higher/lower dose of a test drug. The data from this “N of 1” trial is then used in combination with a database of data for the same drug in like populations in order to add further statistical reliability of the “N of 1” data. More particularly, the results from these microarray or other SNP or proteomic tests will be statistically compared to demographics, disease state, and drug effectiveness/safety to identify correlations. By so doing, one can improve the statistical power of clinical testing kits with newly identified surrogate markers, or even replace the clinical trial effectiveness/safety measurements (when feasible) with tests for genomic/gene expression/proteomic markers in human tissue. The database can also be used to create products to diagnose and treat diseases based on genomic markers, such as SNPs, and gene expression, such as by showing biological predisposition to a disease under defined conditions or by targeting specific classes of drug entities or other interventions for treatment.

One embodiment of the claimed methods involves leveraging the large demographic and clinical effectiveness/safety database obtained from numerous single-patient drug trials by obtaining human biological materials during the trials. It is anticipated that these samples will be, to a large extent, self-funded by cost savings to the health care system. The method will identify surrogate markers of disease etiology/prognosis, drug effectiveness/safety, and/or lifestyle/intervention synergies by testing (as part of each Opt-e-scrip trial) human biological tissue/fluids for testing genetic markers such as microsatellites or Single Nucleotide Polymorphisms (SNPs) using human DNA or RNA microarrays (e.g., chip technology) and successor technologies. Such technologies are exemplified by, for example, micro-array based high capacity SNP multiplexing technologies, (SNP-IT™ marketed by Orchid Biosciences), micro-bead technologies (Megaclone™ and Medasor™ developed by Lynx Therapeutics), mass spectrometric methods (MassARRAY or MassEXTEND systems developed by Sequenom, Inc.), among others. Examples of human biological tissues and fluids to be tested include, but are not limited to, tissue samples, intracellular and extracellular preparations of tissue samples, blood, cerebral spinal fluid, amniotic fluid, bone marrow, visceral fluid, reproductive fluid and excretory fluid.

SNP databases are available for reference purposes, such as the database of the SNP Consortium (A Map of Human Genome Sequence Variation Containing 1.4 Million SNPs, (2001) Nature Vol. 409, pp. 928) and the National Institutes of Health database (dbSNP). Additional information is available on the website of the Human SNP Database: http://www-genome.wi.mit.edu/SNP/human, all of which are incorporated by reference herein. These existing SNP databases make it possible to locate and make reference to common SNPs. Newly discovered SNP targets can be identified on a patient specific basis, and mapped onto the existing SNP map.

Bioinformatics tools can be utilized to aid in data analysis and SNP mapping. Generally, bioinformatics approaches involve sequence analysis using algorithms to detect sequence similarities and identities. Such tools are described in U.S. Pat. Nos. 6,180,358, 6,203,987, and 6,207,373, for example. Searches can be performed, using BLASTN 1.4.9, for example, using a score of 100 and a word length of 12 (Altschul et al. (1990) J. Mol. Biol. 215:403) of the nucleotide sequence of interest, to reveal similarities and sequence identities to known sequences.

In this embodiment of the invention, information derived from testing human biological and fluid samples using human DNA/RNA microarrays or proteomics assays can also be used to screen for changes in gene expression pre- and post-drug treatment. Useful techniques for these tests include, but are not limited to, protein microarrays, DNA and RNA arrays, 2-dimensional electrophoresis, mass spectrometry, etc. The combined gene expression data and SNPs statistical relationships can refine statistical power and predictive capability in future pharmacotherapy optimization and diagnostic products.

This database can also be used to target new drug entities. For example, a patient population can be identified that is susceptible to, or in whom a drug is efficacious, by virtue of the patients' specific SNP makeup. Further study in a patient population having the same SNP makeup allows investigation of new drug entities in a class of drugs which might otherwise appear non-efficacious, or even toxic, when tested in the general population.

There are situations faced by practicing clinicians that require a modification of traditional, fully randomized multiple crossover single-patient drug trials. Specifically, when a patient is placed on or is already using a drug regimen which is believed to be particularly undesirable for chronic use, such as for addictive drugs, a modification of an approach referred to by statisticians as “adaptive allocation” or “play the winner” can be applied.

The test articles to be administered are a less desirable treatment compared to a more desirable alternative treatment. For example, a less desirable drug may be known to cause more toxicity when used over a long period of time compared to the more desirable alternative drug. The initial treatment can be randomized or not randomized. The patient is administered the less desirable drug. If treatment with it succeeds as measured by effectiveness, safety and desirability endpoints, treatment is repeated continued and the endpoints are re-measured. As long as treatment with the less desirable drug succeeds, treatment is continued. If treatment with the less desirable drug fails, the alternative treatment is given. If treatment with the alternative drug succeeds as measured by the same effectiveness, safety and desirability endpoints, treatment is continued and the endpoints are re-measured. If treatment fails, the original, less desirable, treatment is attempted and measured again. As a modification to the method, the regimen can be biased by an attempted “drug holiday” to the safer, more desirable drug if the more dangerous, less desirable drug is repeated routinely, but the physician and patient will continue to be blinded if feasible.

There are situations faced by the practicing clinician that require the ability to predict if a drug of abuse or a drug which may be used therapeutically is likely to have particularly high abuse potential for a specific patient, and/or if the drug is a good candidate for therapeutic use in that patient. Single-patient drug kits can be designed to test for “liking scores,” “abuse potential scores,” and patient's desire to re-use the test article compared to placebo and positive controls. In addition to results from the individual, randomized, double-blinded, multiple crossover single-patient drug trial, population data obtained from previously administered single-patient drug trials in a larger population can be used to improve prediction of abuse potential and appropriateness of the drug for treatment in the individual patient.

Examples of drugs which may be habit forming and possess a high abuse potential include, but are not limited to, nicotine, ethanol, pain medications, sleep aids, diet aids, drugs for treating hyperkinetic behavior, a drug for treating somnolence, a drug for treating anxiety, a central nervous system stimulant, a narcotic analgesic, an anticonvulsant, a sedative-hypnotic, and steroids.

Examples of narcotic analgesics include, but are not limited to the following: alfentanil, allylprodine, alphaprodine, anilerine, benzylmorphine, bezitramide, buprenorphine, butorphanol, clonitazene, codeine, codeine methyl bromode, codeine, desmorphine, dextromoramide, dezocine, diampromide, dihydrocedeine, dihydrocodeinone enol acetate, dihdromorphine, dimenoxadol, dimepheptanol, dimethylthiambutene, dixaphetyl butyrate, dipipanone, eptazocine, ethoheptazine, ethylmethylthiambutene, ethylmorphine, etonitazene, fentanyl, hydrocodone, hydromorphone, hydroxypethidine, isomethadone, ketobemidone, leverphanol, lofentanil, meperidine, meptazinol, metazocine, methodone, metopon, morphine, nyrophine, nalbuphine, narceine, nicomorphine, norlevorphanol, normethadone, normorphine, norpipanone, opium, oxycodone, oxymorphone, papaveretum pentazocine, phenadoxone, phenazocine, phenoperidine, piminodine, piritramide, proheptazine, promedol, propiram, propoxyphene, remifentanil, surfentanil, tilidine, and any salts thereof and mixtures thereof.

Examples of drugs for treating anxiety include, but are not limited to the following benzodiazepines: alprazolam, bromazepam, camazepaam, chlordiazepoxide, clobazam, clorazepate, clotiazepam, cloxazolam, demoxapam, diazepam, ethyl loflazepate, etizolam, fludiazepam, flutazolam, flutoprazepam, halazepam, ketazolam, lorazepam, loxapine, medazepam, metaclazepam, mexazolam, midazolam, nitrazepam, nordazepam, oxazepam, oxazolam, pinazepam, prazepam, tofisopam, and any salts thereof and mixtures thereof.

Examples of sedative-hypnotic agents include, but are not limited to the following: acecarbromal, apronalide, bromisovalum, carbromal, chloral hydrate, glutethimide, chloral betaine, chloral formamide, α-chloralose, chlorhexadol, diethylbromoacetamide, ethchlorvynol, pentaenthritol chloral, mecloqualone, ethaqualone, methyprylon, opium, paradehyde, sulfornethylmethan, sulfon methane, zolpidem, allobarbital, amobarbital, aprobarbital, barbital, brallobarbital, butabarbital, butalbital, butallylonal, butethal, carbubarb, cyclobarbital, cyclopentobarbital, enallylpropymal, 5-furfuryl-5-isopropylbarbituric acid, heptabarbital, hexethal sodium, hexobarbital, mephobarbital, methitural, narcobarbital, nealbarbital, pentobarbital, phenallymal, phenobarbital, phenylmethylbarbituric acid propallylonal, proxibarbal, reposal, secobarbital, talbutal, tetrabarbital, vinbarbital, vinylbital and any salts thereof and mixtures thereof.

Examples of steroids include, but are not limited to the following: boldenone, clostebol, ethylestrenol, fluoxymesterone, formebolone, mesterolone, methandriol, methandrostenolone, methenolone, 17-methyltestosterone, nandrolone, norethandrolone, oxandrolone, oxymesterone, oxymethalone, standone, stanozolol, testosterone, trenbolone, and any salts or mixtures thereof.

Nicotine: Teenagers or other human subjects can be tested for nicotine abuse potential to target likelihood of tobacco addiction and the likelihood of success for nicotine replacement intervention programs. The teenager, possibly a newly discovered smoker, can be subjected to a single-patient drug trial using oral, sublingual, parenteral, inhaled or transdermal nicotine versus placebo. The technique would use a multiple-crossover design, and test for liking scores, desire to re-use the test article, or other measures to test for statistically significant differences.

The results may be included in a larger database, and the patient will be offered behavior modification treatment for prevention of smoking, and followed every 3 months or so to inquire about nicotine addiction. The larger database may be used to decrease statistical variance and increase statistical power for each new individual single patient trial. The database may also be used to feedback outcomes in sub-populations to help the clinician assess the likelihood of abuse based on similar patients.

Ethanol (alcohol): A testing method similar to that above could apply to evaluation of the potential for ethanol abuse. In addition to “liking scores”, additional endpoints could include objective measures, such as EEG measurements.

Pain medications: Human subjects can be tested for opiate abuse potential, targeting the likelihood of opiate addiction and the likelihood for safe and effective use of opiates, such as codeine, propoxyphene, methadone, meperidine, etc. The patient, possibly newly treated for persistent pain (such as headache or back pain), can be subjected to a single-patient drug trial using oral, sublingual, parenteral, inhaled or transdermal drug versus placebo. The technique would use a multiple-crossover design, and test for liking scores, desire to re-use the test article, or employ other measures to test for clinical trends and/or statistically significant differences.

The results may be included in a larger database, the clinician will decide whether or not to prescribe the drug, and the patient will be followed every 3 months to inquire about addiction. The larger database will be used to decrease statistical variance and increase statistical power for each new individual single patient trial. The database will also be used to feed back outcomes in sub-populations to help the clinician assess the likelihood based on similar patients.

Anxiety/Sleep Disorders: Patients with sleep disorders can be tested for the likelihood, for example, of addiction to the sedative/hypnotic class of controlled substances. For example, human subjects can be tested for benzodiazepine or barbiturate addiction potential, targeting the likelihood of addiction and the likelihood for safe and effective use of sedative/hypnotics, such as diazepam, secobarbital, etc. The patient, possibly newly treated for mild to moderate persistent anxiety/sleeplessness with or without pain (such as back pain), can be subjected to a single-patient drug trial using oral, sublingual, parenteral, inhaled or transdermal drug versus placebo. The technique would use a multiple-crossover design, and test for liking scores, desire to re-use the test article, or employ other measures to test for clinical trends and/or statistically significant differences.

The results may be used by the clinician to prescribe or not prescribe the drug. The individual patient data will be included in a larger database, and the patient will be followed every 3 months or so to inquire about addiction. The larger database will be used to decrease statistical variance and increase statistical power for each new individual single patient trial. The database will also be used to feedback outcomes in sub-populations to help the clinician assess the likelihood based on similar patients.

Instead of a placebo, the clinician can use a positive control to test for comparative addiction potential.

### DETAILED DESCRIPTION OF THE PREFERRED EMBODIMENTS

The following non-limiting examples serve to provide further appreciation of the intention but are not meant to restrict the effective scope of the invention. In the examples, a physician usually includes any individual who is licensed or authorized under applicable state law to prescribe prescription drugs.

### EXAMPLE 1

The usefulness of methylphenidate (Ritalin) treatment in a hyperactive child (Minimum Brain Dysfunction or Attention Deficit Disorder) is evaluated.

Rationale: Use of a stimulant in children is highly controversial and widely publicized/perceived as a problem. Parents demand a clear-cut reason to use potentially addictive and often poorly tolerated mediation.

Technology: Consists of instructions, “calendar” packaging for drug and a substantially identical looking placebo to assure appropriate dosing and monitoring of compliance, questionnaires and assessment forms and instructions. Completed forms are sent to a neutral observer who has previously assigned the randomized, multiple-crossover schedule of drug and placebo periods. Only the observer has access to when active drug and placebo are taken and only the observer analyzes the data (exception: open-label or single-blind trials). Results are mailed to the physician and, in this case, parent for use in evaluating the usefulness of the therapy. The physician and parent are contacted, e.g., every three months to provide data on therapies utilized, and perceived outcome, until the condition is resolved. The data is also added to a post-marketing surveillance database for use in evaluating future individual study results, and for access by drug companies, regulatory agencies, and health care organizations.

The questionnaire portion of the kit includes an initial consent form for the parent or guardian to complete. The questionnaire also provides background information on the study and possible side effects associated with the medication. Also included therein is a form for providing relevant patient and family histories. More importantly, the questionnaire, in this case includes a portion for the weekly input by parents/guardians and school observers for answers to questions relating to the drug evaluation. Typical questionnaire sheets for these portions are shown in Table 1 and Table 2 below. Physician questionnaires are similarly arranged.

TABLE 1

| | |
|---|---|
| Safety Net System, Inc. | Hyperactive Child Drug Evaluation Kit |

Week 1

PARENT QUESTIONNAIRE

Date Information Recorded__/__/__{—}

| | |
|---|---|
| Kit Identification Number______{—} | mo day yr |

| | |
|---|---|
| Child's name_________{—} | Parent/Guardian Name_______{—} |

| DEGREE OF ACTIVITY | | |
|---|---|---|
| Place an X on line where appropriate | | |
| OBSERVATION | NOT AT ALL | VERY MUCH |

Restless or overactive, constantly talking, sudden movements (tics), trouble

sleeping
Excitable, impulsive
Disturbs others, fights

Fails to finish things, short attention span, daydreams, won't watch TV for

long
Constantly fidgeting, can't sit still
Inattentive, easily distracted

Demands must be met immediately, easily frustrated, unnecessarily seeks

help

Cries often and easily, sad, fearful, threatens suicide, overly sensitive,

easily hurt, anxious to please, afraid of the dark, has nightmares
Mood changes quickly and drastically
Temper outbursts, explosive and unpredictable

Poor group participation, socially inadequate, isolated, not affectionate,
bullies others, lacks friends, steals, lies, truancy, runs away from home,

destructive, cruel to animals, trouble with police

Defiant, uncooperative, does not recognize authority, talks back, refuses to

obey, fails to return home on time

Abnormal development-clumsiness, speech problems, sexual problems,

abnormal eating habits

To be completed by parent at the end of each study week.

TABLE 2

| | |
|---|---|
| Safety Net System, Inc. | Hyperactive Child Drug Evaluation Kit |

Week 5

SCHOOL QUESTIONNAIRE

Date Information Recorded__/__/__{—}

| | |
|---|---|
| Kit Identification Number______{—} | mo day yr |

| | |
|---|---|
| Child's name_________{—} | School Observer Name______{—} |

| DEGREE OF ACTIVITY | | |
|---|---|---|
| Place an X on line where appropriate | | |
| OBSERVATION | NOT AT ALL | VERY MUCH |

Restless or overactive, leaves seat unexcused, nervous, tense

Excitable, impulsive
Disturbs other children, fights, noisy, tapping, humming
Fails to finish things, short attention span
Constantly fidgeting
Inattentive, easily distracted

Demands must be met immediately, easily frustrated, speaks out

of turn

Cries often and easily, sad, sullen, overly sensitive, easily hurt,

anxious to please
Mood changes quickly and drastically
Temper outbursts, explosive and unpredictable
Poor group participation, socially inadequate, isolated
Defiant, uncooperative, does not recognize authority

Abnormal development-bedwetting, clumsiness, speech

problems, sexual problems, abnormal eating habits

To be completed by school observer at the end of each study week.

As a result of undergoing the study, all interested parties have a clear understanding of the value of the medication for this particular patient.

### EXAMPLE 2

The kit described in Example 1 is used to again evaluate the usefulness of methylphenidate (Ritalin) treatment in a hyperactive child except that all interested parties have the benefit of a set of data generated from a pool of patients having a similar need for treatment. The trial calls for 40 mg to be given daily as 10 mg four times daily compared to identical appearing placebo which is also given four times daily. After completing the trial and questionnaire, the data is processed to statistically determine the results. The results are provided as follows:

the patient's attention span is observed to have improved substantially during the periods in the trial when the methylphenidate is being given;

temper outbursts are observed to increase slightly during placebo periods;

sleep patterns are observed to be statistically altered during methylphenidate periods; and

teacher comments corroborate improved attention span during methylphenidate dosing periods.

All results obtained from the data are compared against the results provided by data amassed from a pool of about 200 patients with the same disorder. If, of these 200 patients, 55 experience encouraging results (along with the current patient) they are continued on 40 mg daily treatment. If, of these 55 patients, 5 are lost to follow-up, with 50 remaining for prospective evaluation, the physician continues the patient on 40 mg methylphenidate daily based solely on the isolated SPAS single-patient drug trial results. The physician then reviews the pooled data on the 50 patients. This will be done to understand under what circumstances this individual patient is likely to continue to show benefit from methylphenidate 40 mg treatment, and what conditions lead to treatment failure.

If for example, the pool of 50 patients who continue treatment have the following scores following the original SPAS testing:

| | |
|---|---|
| Attention Span | 100% have substantial improvement |
| Sleep Patterns | 50% have been statistically altered |
| | 50% have not been statistically altered |
| Teacher Comments | 80% corroborate improved attention span |
| | 20% do not corroborate improved attention span |

All 50 patients are followed up by telephone interview monthly for nine months or more, and outcomes are prospectively documented. If it is found that all patients who have no statistically altered sleep disturbances on the original SPAS test continue to be well maintained on treatment, then their treatment remains the same. However, if within two months, all patients who have statistically altered sleep patterns on SPAS testing show loss of symptom control, e.g., 90% show severe episodes of bizarre behaviors; two patients experience grand mal seizures, these patients having statistically altered sleep disturbances are discontinued from treatment within two months.

Despite an initial, generally positive result of the SPAS single-patient drug trial, the prescribing physician has a strong, objective basis for not continuing treatment with methylphenidate 40 mg daily because the pooled data from similar patients clearly indicates that continued treatment in the presence of sleep alteration is a great risk (e.g.,90% chance of a severe adverse event) with limited potential benefit. The continuing validation process using pooled data, which is a subject of this invention, provides additional data essential to formulating a rational therapeutic decision.

The physician now decides to use a different pharmacologic intervention in a chemical class which is not as frequently associated with sleep disturbance (e.g., amitriptyline), or decides to use non-pharmacologic treatments, such a behavioral therapy. The amitriptyline dose selected is tested using another SPAS designed for that drug and the process is continued until the patient is on a documented safe and effective drug regimen.

The usefulness and practicality of the method of doing business which comprises the use of a single-patient clinical trial flowchart is better understood when analyzed under the aforementioned examples.

In Examples 1 and 2, the usefulness of methylphenidate (Ritalin) in the treatment of a hyperactive child is evaluated. Example 2 includes the further benefit of comparing the results obtained from the objective questionnaires filled-out by the patient/physician with a set of data generated from a pool of patients having a similar need for treatment. The use of the flowchart in these examples provides methods of optimizing the clinical outcome, providing rational (evidence-based) pharmacotherapy, and decreasing healthcare costs.

The step-by-step analysis using the flowchart for determining the usefulness of methylphenidate (Ritalin) in the treatment of a hyperactive child is as follows:

Step 1: the child is evaluated to determine whether he/she is a candidate for the clinical trial. If the child is a qualified candidate, the child is evaluated using Step 2.

Step 2: the physician considers whether a) the child qualifies to receive a high cost and low cost alternative drug; (b) no low cost alternative drug is available; (c) the child is already taking drug, but effectiveness or safety vs. placebo is unknown; (d) the child is already taking drug, but an optimal dose is uncertain; or (e) the child is taking drug, but a less expensive alternative drug can be considered.

a) When the child qualifies to receive a high cost and low cost alternative drug, he/she receives methylphenidate and some other drug (one of which is more costly than the other).

Step 3: the child is assigned to receive a Switchability test kit which tests the methylphenidate vs. the other drug and, optionally vs. placebo.

Step 4: the effectiveness and safety of the methylphenidate, other drug, and placebo are determined. If the methylphenidate and the other drug and placebo have comparable effectiveness and safety results, then the physician prescribes the less expensive drug. If effectiveness and safety remains beneficial for only one drug, the physician prescribes that drug.

b) When no low cost alternative drug is available or when the child is taking drug, but effectiveness and safety vs. placebo is unknown, he/she qualifies to receive methylphenidate and placebo.

Step 3: the child is assigned to receive a Prescribability test kit which tests the methylphenidate vs. placebo.

Step 4: the superiority of the methylphenidate vs. placebo is determined. If the methylphenidate is more acceptable than placebo, the physician prescribes the methylphenidate. If the placebo is more acceptable or equivalent to the methylphenidate, the physician prescribes placebo or an alternative therapy such as a low risk active drug or dietary supplement, which in turn decreases the costs of therapy.

c) When the child is taking drug, but an optimal dose is uncertain, he/she qualifies to receive methylphenidate at a higher and lower dose.

Step 3: the child is assigned to receive a Dosability test kit which tests the methylphenidate at a high and low dose.

Step 4: the overall profile of the high dose of methylphenidate is compared to the low dose of methylphenidate. If the high dose of methylphenidate is more acceptable than (has a better overall profile) the low dose of methylphenidate, the physician prescribes the higher dose. If the low dose of methylphenidate is more acceptable than the high dose of methylphenidate, the physician prescribes the lower dose.

d) When the child is taking drug, but a less expensive alternative drug is being considered, he/she qualifies to receive methylphenidate and the less expensive alternative.

Step 3: the child is assigned to receive a Switchability test kit which tests the methylphenidate vs. the less expensive alternative drug.

Step 4: the effectiveness and safety of the methylphenidate and the less expensive alternative drug are determined. If the methylphenidate and the less expensive alternative drug have comparable effectiveness and safety results, then the physician prescribes the less expensive alternative drug. If effectiveness and safety is more acceptable for only one drug, the physician prescribes that drug.

Step 5: the child is prescribed a specific treatment regimen, and further safety, efficacy and desirability data is collected. If the safety, effectiveness and desirability remain the same, then the child remains on the specific treatment prescribed. Should the safety, effectiveness and desirability of the treatment deteriorate or a fixed interval elapses, requiring re-evaluation of treatment (drug holiday), then the physician/caretaker must target other appropriate alternative drugs and timings for additional single-patient trials including drug holidays and re-tests (Step 6).

Step 6: is a last resort whenever the child's treatment becomes ineffective, unsafe or unsubstantiated, whether it be the methylphenidate, another alternative drug or placebo. Step 6 involves targeting alternative drugs and timings for other single-patient trials which include drug holidays and re-tests.

### EXAMPLE 3

Test kit: Antihistamine for house dust induced allergic nasal congestion.

A clinician writes a prescription for a test kit which has been extensively tested in patients similar to his. The product labeling available to the clinician advises him that it has been used in 2,000 patients with house dust allergic nasal congestion to date. The antihistamine is found to be clinically useful with a modest side effect profile in 1500 patients, 250 experience untoward drowsiness and 250 experience no clinical benefit. The test is completed and found useful only in subjects with an 8^{th} grade educational level or higher who report at least moderate symptom on study initiation. Subjects with mild symptoms often fail to complete the study. The clinician recognizes that this patient is college educated with severe symptoms and writes the prescription, confident that he has a good candidate for the test.

### EXAMPLE 4

The pharmaceutical company marketing an antihistamine submits a 2,000 patient database to the government for approval of a new claim for the product: house dust allergic nasal congestion. The company agrees with a request from the government agency that, as a condition for expedited review and acceptance, continuous post-marketing surveillance will be conducted for this indication by marketing the product in a SPAS test kit. This testing of each subject on initiation of therapy will continuously ensure that each patient is evaluated for appropriateness of treatment prior to commitment to a chronic regimen. In addition, it allows the company to provide a monthly update to the government of drug effectiveness and safety in the entire population using the product for house dust allergic nasal congestion. Physician and patient labeling is revised when necessary. Also, it is a way of finding out whether more side effects occur when the product is concomitantly taken with another drug (e.g., cimetidine). Therefore, the company now advises the government agency of any possible drug interaction, and warns clinicians of a possible drug interactions in the product labeling.

### EXAMPLE 5

The use of Claritin^{R} (loratidine-CL) is restricted on a managed care formulary, for example, because of high cost relative to generic chlorpheniramine maleate (CM) for treatment of allergic rhinitis. This is because only a small subset of the population experiences untoward sedation while taking CM during chronic treatment, and therefore it is often unnecessary to use an expensive non-sedating antihistamine, such as CL. The existing formulary management system prohibits initial or routine use of CL and penalizes use of CL with a higher co-pay cost to the patient.

An alternative is to encourage use of a single-patient drug trial comparing CL to CM. The individual patient, for example, is included in the trial if previous single-patient trials indicate that their history, demographics and illness are compatible with execution of the trial. Their incentive, such as co-pay amounts, is determined by willingness to participate, and later, by outcome of the trial.

For example, a 28 year old Caucasian female of Irish ancestry with known dust mite protein allergy living on the West side of Manhattan presents for initial treatment of perennial allergic rhinitis. It is determined that she is a candidate for the individual patient formulary management control system. She is considered a candidate because there are similar patients, e.g. 50, in the database, and 40 of them are placed on CM rather than CL based upon the outcome of single-patient drug trials. Thirty-seven (37) of the 40 are well maintained on CM during a year long follow-up, and two are later switched to CL and one discontinues treatment due to spontaneous resolution of symptoms. The current patient is administered a single-patient drug trial according to the present invention. The statistical power is enhanced by applying a pooled estimate of variance from previous single-patient drug trials. It is found that her most bothersome symptom, sneezing, is similarly and effectively controlled by both CL and CM, she is prescribed the less expensive CM. It is found that the incidence of sedation on day 4 of treatment is 2 on a 3 point scale for both CL and CM, based on these results, CM is prescribed. CL is only prescribed under these circumstances at a higher co-pay under an individually determined formulary management control system. If CL is found to be more acceptable considering the safety, effectiveness and desirability data of the individual patient questionnaire, the co-pay is the same low rate as for CM. At 6 and 12 months post-initiation of CM treatment the patient is reevaluated. The patient's symptoms are well maintained, and she is continued on the same treatment. This information is added to the database to improve the estimate of variance for subsequent single-patient drug trials. The managed care organization saves significantly by using the inexpensive drug, the patient's care is not compromised and, in fact, is demonstrated to be excellent.

The step-by-step analysis for using the flowchart to evaluate the use of an antihistamine for: 1) the treatment of house dust induced allergic nasal congestion; 2) conducting continuous post-marketing surveillance; or 3) comparing the effectiveness and safety of two different antihistamines is as follows:

Step 1: a patient is evaluated to determine whether the patient is a candidate for the clinical trial.

Step 2: the physician determines whether a) the patient qualifies to receive a high cost and low cost alternative drug; (b) no low cost alternative drugs are available; (c) the patient is taking drug, but effectiveness or safety vs. placebo is unknown; (d) the patient is taking drug, but an optimal dose is uncertain; or (e) the patient is taking drug, but a less expensive alternative drug can be considered.

a) When a patient qualifies to receive a high cost and low cost alternative drug, the patient receives a higher cost antihistamine (e.g., Claritin, Hismanal) and some other lower cost antihistamine (e.g., chlorpheniramine, diphenhydramine).

Step 3: the patient is assigned to receive a Switchability test kit which tests the higher cost antihistamine (Claritin) vs. the lower cost antihistamine (diphenhydramine) and, optionally, vs. placebo.

Step 4: the effectiveness and safety of the Claritin, diphenhydramine, and, optionally, placebo is determined, assuming that if placebo is used, either drug is better than placebo. If the Claritin and diphenhydramine have comparable effectiveness and safety results, the physician prescribes the diphenhydramine (less expensive alternative). If effectiveness and safety remains beneficial for only one of the treatments e.g., Claritin, the physician prescribes the Claritin.

b) When a patient is new with no low cost alternative drug, the patient qualifies to receive a high cost antihistamine (Claritin) and placebo.

Step 3: the patient is assigned to receive a Prescribability test kit which tests the Claritin vs. placebo.

Step 4: the superiority of the Claritin vs. placebo is determined. If the Claritin is more acceptable than placebo, the physician prescribes the Claritin. If the placebo is more acceptable or equivalent to the Claritin, the physician prescribes the placebo or a low risk therapeutic agent or herbal remedy, which in turn decreases the costs of therapy.

c) When a patient is taking drug e.g., Claritin, but effectiveness and safety vs. placebo is unknown, the patient qualifies to receive the Claritin they were currently receiving and placebo.

Step 3: the patient is assigned to receive a Prescribability test kit which tests the Claritin the patient is receiving vs. placebo.

Step 4: the superiority of the Claritin vs. placebo is determined. If the Claritin is more acceptable than placebo, the physician continues to prescribe the Claritin the patient is receiving. If the placebo is more acceptable or equivalent to the Claritin, the physician prescribes the placebo or a low risk therapeutic agent or herbal remedy, which in turn decreases the costs of therapy.

d) When a patient is taking drug, e.g., Claritin, but an optimal dose is uncertain, the patient qualifies to receive the Claritin at a higher and lower dose.

Step 3: the patient is assigned a Dosability test kit which tests the Claritin at a high and low dose.

Step 4: the overall profile of the high dose of Claritin is compared to the low dose of Claritin. If the high dose of Claritin is more acceptable than the low dose of Claritin, the physician prescribes the higher dose. If the low dose of Claritin is more acceptable than the high dose of Claritin, the physician prescribes the lower dose.

e) When a patient is taking drug, e.g., Claritin, but a less expensive alternative drug can be considered, e.g., diphenhydramine, the patient qualifies to receive Claritin and the less expensive alternative, diphenhydramine.

Step 3: the patient is assigned a Switchability test kit which tests the Claritin the diphenhydramine.

Step 4: the effectiveness and safety of the Claritin and the diphenhydramine are determined. If the Claritin and diphenhydramine have comparable effectiveness and safety results, then the physician prescribes the diphenhydramine. If the effectiveness and safety are more acceptable for only one of the treatments, the physician prescribes the more acceptable treatment.

Step 5: the patient is prescribed a specific treatment regimen, and further safety, effectiveness and desirability data is collected. If the safety, effectiveness and desirability remain the same, then the patient remains on the specific treatment prescribed. Should the safety, effectiveness and desirability of the treatment deteriorate or a fixed interval elapses, requiring re-evaluation of treatment (drug holiday), then the physician/caretaker must target other appropriate alternative drugs and timings for additional single-patient trials including drug holidays and re-tests (Step 6).

Step 6: is a last resort whenever the patient's treatment becomes inefficacious or unsafe, whether it be the Claritin, another alternative drug or placebo. Step 6 involves targeting alternative drugs and timings for other single-patient trials which include drug holidays and re-tests.

### EXAMPLE 6

In this example, the validity of using a sustained release formulation of verapamil 240 mg once daily as a therapeutic alternative to sustained release propranolol 180 mg once daily in a hypertensive 45 year old male is shown. The trial during which the two medications are randomly administered in a double-blind manner is six weeks. The questionnaire used is set up in a manner similar to that described in above examples except that the questions elicit information related to the disease of hypertension. Blood pressure and adverse event reports are taken daily by the patient. At the end of the trial period it is determined that mean systolic blood pressure increases 3% and mean diastolic pressure is essentially stable throughout the trial and there is little difference between drugs. Sleepiness is reported at equal rates for both drugs. All values are statistically insignificant and the therapeutic alternative selection is objectively validated.

### EXAMPLE 7

In this example, the data is accumulated from example 6 and compared against that acquired from a pool of 100 male patients who were switched from beta blockers, including propranolol, to calcium channel blockers, including verapamil. The results of example 6 are found to be in agreement with those found from the pool. On this basis, a health maintenance group can objectively recommend the use of beta blockers for males fitting the pool profile with hypertension under its care, if they will not participate in SPAS for definitive data.

The step-by-step analysis using the flowchart for determining the validity of using a sustained release formulation of verapamil (240 mg) once daily as a therapeutic alternative to sustained release propranolol 180 mg once daily in a hypertensive 45 year old male is as follows:

Step 1: the patient is evaluated to determine whether the patient is a candidate for the clinical trial.

Step 2: is by-passed because the patient will automatically receive a Switchability test kit.

Step 3: the patient is assigned to a Switchability test kit which tests verapamil vs. propranolol, and optionally, vs. placebo.

Step 4: the effectiveness and safety of the verapamil, propranolol, and placebo are determined. If the verapamil and the propranolol and placebo have comparable effectiveness and safety results, the physician prescribes the less expensive drug. If effectiveness and safety is more acceptable for only one of the treatments, e.g., verapamil, the physician prescribes the verapamil.

Step 5: the patient is prescribed verapamil, and further safety, efficacy and desirability data is collected. If the safety, effectiveness and desirability remain the same, then the patient remains on the verapamil. Should the safety, effectiveness and desirability of the verapamil treatment deteriorate, then the physician/caretaker targets other appropriate alternative drugs and timings for additional single-patient trials including drug holidays and re-tests (Step 6).

Step 6: is a last resort whenever the patient's treatment becomes inefficacious or unsafe, whether it be the verapamil, another alternative drug or placebo. Step 6 involves targeting alternative drugs and timings for other single-patient trials which include drug holidays and re-tests.

A similar analysis occurs when Example 7 is plugged into the flowchart.

### EXAMPLE 8

In this example, each member of a pool of fifty patients is given a test kit containing a sixteen day supply of an antihistamine and a sixteen day supply of a look-alike placebo arranged in a multiple-crossover, four-days each study leg, eight days each crossover, randomly ordered design, along with a questionnaire designed to confirm the appropriateness of the therapy. After all of the kits are finished and individual results are provided to the patients and care-givers, the pooled data supplied from the questionnaire is evaluated. If it is found that a question (e.g., number 12) relating to the secondary side-effect of dry mouth for the drug is poorly understood by the pool members and fails to provide a statistically significant result for the database and, moreover, several patients report heart palpitations and related cardiac disturbances, question number 12 is dropped from the questionnaire and replaced with one tested and validated for comprehension at the 8^{th} grade education level; also a new question relating specifically to cardiac symptoms is added. All further kits for the antihistamine trials are made to contain the revised, validated questionnaire. A clinician writes a prescription for a test kit containing the revised questionnaire and antihistamine/placebo combination for a patient. The patient completes the course of therapy as directed over the eight week course and completes the weekly questionnaire relating to the trial and mails them to a neutral observer who also has the key to the random arrangement of drug/placebo. At the end of the trial, a statistical analysis of the trial is provided to the clinician who evaluates the results in view of the data provided by the pool of patients. The clinician thus has an objective basis for continuing the therapy since this individual is found to have substantially improved symptoms, and members of the tested pool with similar results are usually found to do well initially with continued treatment at three and six months.

As will be readily appreciated, numerous variations and combinations of the features set forth above can be utilized without departing from the present invention as set forth in the claims. Such variations are not regarded as a departure from the spirit and scope of the invention, and all such modifications are intended to be included within the scope of the following claims.

In Example 8, Step 2 of the single-patient clinical trial flowchart is by-passed.

Step 3: the patients are automatically assigned to receive a Prescribability test kit (active vs. placebo) wherein each individual member of a pool of 50 patients is given a test kit containing a sixteen day supply of an antihistamine and a sixteen day supply of a look-alike placebo. The superiority of the antihistamine vs. placebo is evaluated. If the antihistamine is more acceptable than placebo, the physician prescribes the antihistamine. If the placebo is more acceptable than the antihistamine, the physician prescribes placebo, or low risk therapeutic agent (which may include an herbal remedy).

In addition, Example 8 provides a method of providing a more effective single-patient test kit. For example, the superiority of the antihistamine vs. placebo is evaluated. The results of the evaluation are obtained from the data supplied in the questionnaire. The questions relate to side-effects, reduction of symptoms, etc. . . . , which are objectively supplied by the patient and physician. In certain situations, poorly understood questions are dropped from the questionnaire and replaced with other tested and well understood questions. These revised validated questionnaires in turn then provide a more effective single-patient test kit.

### EXAMPLE 9

In this example a Single-Patient Assessment System (SPAS) is utilized to optimize chronic treatment in an individual patient with a drug determined to be useful for the treatment of glaucoma, a disease state which has been identified by specific genetic markers (SNP's).

A patient diagnosed with glaucoma is assigned a SPAS test kit containing two drugs commonly prescribed for the treatment of glaucoma (timolol and pilocarpine), and questionnaires and assessment forms for the collection of data during the trial. The patient receives one (1) drop of timolol 0.25% ophthalmic solution two times daily and one (1) drop of pilocarpine 0.5% ophthalmic solution three to four times daily in a randomized, crossover Id manner for a total of 8 weeks. The questionnaire is set up to elicit information related to the disease of glaucoma. Eye exams are conducted weekly by the ophthalmologist. Patient's biological fluids (e.g., saliva, blood) or tissues (e.g., epithelial cell samples, endothelial cell samples or hair) are collected to determine if the FKHL7 genetic marker for glaucoma is present. Evaluation of biological fluids to determine if the specific genetic marker is present is accomplished using the SNP and Microarray technology previously discussed.

Once the specific genetic marker is identified in the patient, the physician compares the results from the data collected in the single patient trial against a pooled database of similar conducted N of 1 trials in patients having the same genetic marker for glaucoma. The comparison is used by the physician to optimize the individual patient's drug therapy based on the successes and failures of the different treatments in the pooled database of N of 1 trials. The results of the individual single patient trial are then added to the pooled database of N of 1 trials which was used to compare and optimize the individual patient's therapy.

### EXAMPLE 10

A Single-Patient Assessment System (SPAS) is utilized to modify treatment of a patient suffering from a sleep disorder. The patient receives a SPAS test kit containing a less desirable, more toxic drug treatment, e.g., Valium^{R} (diazepam, a controlled substance of the benzodiazepine class) for anxiety/sleep disorder, a more desirable, safer comparative drug, e.g., Benedryl^{r} (diphenhydramine, a non-addictive antihistamine) with known ability to induce sleep and a questionnaire designed to elicit from the patient data concerning the safety, effectiveness and desirability of the two drug treatments. The drug treatments are administered in a randomized double-blind or single-blind manner. If, for example, the diphenhydramine is initially administered because it is the safer agent, it is measured for its effectiveness, safety and desirability. If its safety, effectiveness and desirability is acceptable, the patient is continued on this agent until it fails. If the safety, effectiveness and desirability of the diphenhydramine is not found acceptable, diazepam is then administered. The safety, effectiveness and desirability of the diazepam is measured. If the safety, effectiveness and desirability of diazepam is acceptable, treatment is continued until it fails, in which case treatment with diphenhydramine is re-attempted and repeated as long as it succeeds. Every (e.g.—ninth, tenth, or eleventh) treatment with diphenhydramine is attempted to assure that the safer agent is attempted on a routine basis. The clinician/patient knows that an attempt is made on a regular basis to switch to the safer drug, but they do not know on which day or time this is tried. Other approaches to bias towards the safer agent are also attempted as a deviation from the traditional “adaptive allocation” or “play the winner” statistical method.

Example 10 provides a method for modifying traditional, fully randomized multiple crossover single-patient drug trials, specifically when a patient is placed on or is already using a drug regimen which is undesirable for chronic use, e.g., addictive drugs.

The single-patient clinical trial flowchart is used to evaluate a patient receiving Valium® for anxiety or sleep disorders as follows:

Step 1: the patient is evaluated to determine whether the patient qualifies as a candidate for the clinical trial.

Step 2: evaluates whether a) the patient qualifies to receive a less desirable, more toxic drug treatment and a more desirable, safer comparative drug; (b) no more desirable, safer comparative drug is available; (c) patient is taking less desirable drug, but effectiveness or safety vs. placebo is unknown; (d) taking less desirable drug, but an optimal dose is uncertain; or (e) taking less desirable drug, but a less expensive, safer comparative drug can be considered.

a) When a patient qualifies to receive treatment with a less desirable, more toxic drug treatment and a more desirable, safer comparative drug, the patient qualifies to receive Valium® and some other drug used to treat anxiety or sleep disorders, e.g., diphenhydramine.

Step 3: the patient is assigned to receive a Switchability test kit which tests the Valium® vs. the diphenhydramine, and optionally, vs. placebo.

Step 4: the effectiveness and safety of the Valium®, diphenhydramine, and optically, placebo are determined using a “play the winner” comparison. If diphenhydramine or placebo have satisfactory effectiveness and safety results, then the physician prescribes the safer and often less expensive drug, e.g., diphenhydramine. Once the patient receives the diphenhydramine, the effectiveness and safety are continuously monitored. If the safety and effectiveness of the diphenhydramine are not acceptable, the alternative medicine, e.g., Valium® is prescribed. The safety and effectiveness of the alternative treatment is measured, and if acceptable, is repeated and re-measured. If the safety and effectiveness of the alternative treatment is unacceptable, then the original treatment, e.g., diphenhydramine, is attempted and measured again. This regimen is also modified by attempting a “drug holiday” to the safer drug if the more dangerous drug is routinely repeated.

b) When no more desirable, safer comparative drug is available or c) when patient is taking less desirable drug, but effectiveness or safety vs. placebo is unknown, the patient qualifies to receive Valium® and placebo.

Step 3: the patient is assigned to receive a Prescribability test kit which tests the Valium® vs. placebo.

Step 4: the safety and effectiveness of the Valium® vs. placebo is determined. If the safety and effectiveness of Valium® is more acceptable than placebo, the physician prescribes the Valium®. If the safety and effectiveness of placebo is more acceptable or equivalent to the Valium®, the physician prescribes the placebo or a low risk therapeutic agent, which includes herbal remedies, which in turn will decrease the costs of therapy.

d) When a patient is taking less desirable drug, but an optimal dose is uncertain, the patient qualifies to receive Valium® at a higher and lower dose.

Step 3: the patient is assigned to receive a Dosability test kit which tests the Valium® at a high and low dose.

Step 4: the overall profile of the high dose of Valium® is compared to the low dose of Valium®. If the safety and effectiveness of the high dose of Valium® is more acceptable than the low dose of Valium®, the physician prescribes the higher dose. If the safety and effectiveness of the low dose of Valium® is more acceptable than the high dose of Valium®, the physician prescribes the lower dose.

e) When a patient is taking less desirable drug, but a less expensive, safer comparative drug is considered, the patient qualifies to receive Valium® and the less expensive alternative.

Step 3: the patient is assigned to receive a Switchability test kit which tests the Valium® vs. the less expensive alternative drug.

Step 4: the effectiveness and safety of the Valium® and the less expensive alternative drug are determined. If the Valium® and the less expensive alternative drug have comparable effectiveness and safety results, then the physician prescribes the less expensive alternative drug. If effectiveness and safety remains beneficial for only one of the treatments, the physician prescribes the more acceptable treatment.

Step 5: the patient is prescribed a specific treatment regimen, and further safety, effectiveness and desirability data is collected. If the safety, effectiveness and desirability remain the same, then the patient remains on the specific treatment prescribed. Should the safety, effectiveness and desirability of the treatment deteriorate or a fixed interval elapses requiring re-evaluation of treatment (drug holiday), then the physician/caretaker targets other appropriate alternative drugs and timings for additional single-patient trials including drug holidays and re-tests.

Step 6 is a last resort whenever the patient's treatment becomes ineffective, unsafe or unsubstantiated, whether it be the Valium®, another alternative drug or placebo. Step 6 involves targeting alternative drugs and timings for other single-patient trials which include drug holidays and re-tests.

### EXAMPLE 11

Utilizing a Single-Patient Assessment System (SPAS) a physician can test for the abuse potential of a drug determined useful for the treatment of a specific disease or symptom. In this example, the abuse potential of the narcotic analgesic codeine is tested. A single-patient trial is conducted in a patient for whom treatment with codeine is deemed appropriate. The patient receives a SPAS test kit containing codeine, an alternative drug, e.g., ibuprofen and a questionnaire designed to elicit the liking score, desirability to re-use test article and abuse potential of the patient to codeine. For example, a liking score of 3 out of 5 is found, in a 45-year-old black male patient on codeine. A liking score of 1 out of 5 is found for placebo. The difference of 2 units is found to be statistically significant with statistical feedback from previously tested subjects using Bayesian techniques to decrease statistical variance via feedback from previously tested patients. It is also found that there were many other black male patients who were between 40 and 50 years old at the time of testing; these have a similar 2-unit difference and were treated with codeine. Of these, only 25 out of 600 became addicted. The clinician concludes that the risk of this new patient becoming addicted is modest despite the statistically significant result.

In another example, a liking score of 4 out of 5 is found for a 34-year-old Caucasian female patient on diazepam. Four (4) out of 5 is also found for placebo. The difference of 0 units is found to not be statistically significant with or without statistical feedback from previously tested subjects using Bayesian techniques to decrease statistical variance via feedback from previously tested patients. It is also found that there were many other Caucasian female patients who were between 30 and 40 years of age at the time of testing; 1250 of these have a similar 0 unit difference and were treated with codeine. Of these, only 10 out of 1250 became addicted. Twenty out of 105 of similar patients with 2 unit differences became addicted within three months, and 50 out of 110 with 3 unit differences became addicted. The clinician concludes that the risk of this new patient becoming addicted is modest and that the risks have been adequately validated.

In another example, in follow-up to the test above, the clinician compares diazepam to secobarbital in the same 34-year-old Caucasian female patient. A statistically significant difference in liking scores showing greater addiction potential for the secobarbital guides the clinician to prescribe diazepaam. This decision is particularly compelling because a high level of addiction to secobarbital is observed on follow-up of similar patients, and a comparatively low level of addiction is found for diazepam.

In yet another example, a “liking score” of 4 out of 5 is found, on average, for a 16-year-old Caucasian female patient receiving nicotine. One (1) out of 5 is found for placebo. The difference of 3 units is found to be statistically significant. Using a Bayesian statistical approach that decreased the statistical variance via feedback from previously tested patients, the p value is less than 0.02. Using a frequent approach, it is less than 0.05. Without the feedback from the larger population, the p value is 0.10. Because it is also found that for other female Caucasian patients who are between 15 and 17 years old at the time of testing and had a similar 3-unit difference, 820 out of 975 are smokers. The clinician concludes that the risk of this new patient becoming a smoker is large based on these results.

While there have been described what are presently believed to be the preferred embodiments of the invention, those skilled in the art will realize that changes and modifications may be made thereto without departing from the spirit of the invention. It is intended to claim all such changes and modifications that fall within the true scope of the invention.

What is claimed is:

1. A method of optimizing clinical outcomes and providing pharmacotherapy in an individual human patient for whom chronic drug therapy is contemplated, comprising: a) determining a first drug for treatment of an individual human patient for whom chronic drug therapy is contemplated, and a second drug which may alternatively be useful for treatment of the individual human patient; b) conducting a single patient cross-over drug trial in the individual human patient via a switchability test utilizing a supply of the first drug; a supply of the second drug, and optionally a supply of placebo; and accumulating information concerning the safety, effectiveness, patient compliance and desirability of the first drug, the second drug and optionally the placebo; c) evaluating whether safety, effectiveness, patient compliance and desirability is acceptable for both the first drug and the second drug; one of the first drug and the second drug; or neither the first drug or the second drug, optionally as compared to the placebo, by comparing the results of the single patient drug trial of the individual human patient with a previously assembled patient population database of information concerning the safety, effectiveness, patient compliance and desirability of the first drug, the second drug and optionally the placebo administered in a plurality of cross-over single patient drug trials, to aid in the interpretation of the results for the new patient; and d) optimizing treatment for the patient by taking one of the following actions: (i) if safety, effectiveness, patient compliance and desirability is acceptable for both the first drug and the second drug, initiating chronic therapy for the individual human patient using the first drug or the second drug, taking into account the relative benefits of each drug based on the results of the evaluation of safety, effectiveness, patient compliance and desirability of the first drug and the second drug as compared to the patient population database, as well as the relative cost of the first drug and the second drug; (ii) if safety, effectiveness, patient compliance and desirability are acceptable for only one of the first drug and the second drug, initiating chronic therapy for the individual human patient using the acceptable one of the first drug or the second drug; (iii) if safety, effectiveness, patient compliance and desirability are not acceptable for either of the first drug and the second drug, discontinuing treatment or repeating steps (b)-(d) utilizing third and fourth alternative drugs, if available.

2. The method of claim 1, further comprising assembling said patient population database from a plurality of cross-over single patient drug trials prior to conducting step (a).

3. The method of claim 1, further comprising adding the results from the single patient drug trial of the individual human patient to the patient population database.

4. The method of claim 1, further comprising accumulating the information of step (b) via the use of objective testing methodologies selected from the group consisting of blood pressure, cholesterol, blood sugar, glycosylated hemoglobin and combinations of any of the foregoing.

5. The method of claim 1, further comprising including a questionnaire in said test, said questionnaire designed to elicit from said individual patient or caretaker information concerning the actual usage, safety, effectiveness and desirability of said drug and said second agent.

6. The method of claim 1, further comprising prescribing said first drug for chronic therapy in said patient.

7. The method of claim 1, further comprising prescribing said second drug for chronic therapy in said patient.

8. The method of claim 1, wherein said patient population database is stored on a computer.

9. The method of claim 8, wherein said computer database is accessible from a remote location.

10. The method according to claim 1, wherein said drug or said second drug is selected from the group consisting of a drug for treating hyperkinetic behavior, cancer, schizophrenia, minimal brain dysfunction, mania, alzheimer's disease, attention deficit disorder (ADD), angina, congestive heart failure, cardiac arrhythmias, pain, metabolic disorders, endocrine disorders, obesity, neurologic disorders, immunologic diseases, eye disorders, ear disorders, sleep disorders, central nervous system disorders, urinary tract disorders, renal disorders, genito-urinary disorders, erectile dysfunction, podiatric disorders, chiropractic disorders, geriatric conditions, anti-asthmatic agents, dental agents, anti-epileptic agents, anti-psychotic agents, anti-depressants, cardiovascular agents, respiratory agents, neurological agents, antihypertensive agents, diabetic agents, steroidal and non-steroidal anti-inflammatory agents, opiates, narcotic and non-narcotic analgesics, hematologic agents, musculoskeletal agents, anti-anxiety agents, gastro-intestinal agents, dermatologic agents; and anti-allergy medications.

11. The method according to claim 1, wherein said switchability test comprises a supply of a drug therapy; a supply of a second drug therapy; and a questionnaire designed to elicit from said individual or caretaker information concerning the actual usage, safety, effectiveness and desirability of said drug therapy and said second drug therapy.

12. A method of optimizing clinical outcomes and providing pharmacotherapy in an individual human patient for whom chronic drug therapy is contemplated, comprising: a) determining a drug for treatment of an individual human patient for whom chronic drug therapy is contemplated; b) conducting a single patient cross-over drug trial in the individual human patient via a prescribability test utilizing a supply of the drug and a supply of a placebo; and accumulating information concerning the safety, effectiveness, patient compliance and desirability of the drug, and the placebo; c) evaluating whether safety, effectiveness, patient compliance and desirability is more acceptable for the drug than the placebo; more acceptable for the placebo than the drug; or equivalent for both the drug and the placebo, by comparing the results of the single patient drug trial of the individual human patient with a previously assembled patient population database of information concerning the safety, effectiveness, patient compliance and desirability of the drug and the placebo administered in a plurality of cross-over single patient drug trials, to aid in the interpretation of the results for the new patient; and d) optimizing treatment for the new patient by taking one of the following actions: (i) if safety, effectiveness, patient compliance and desirability are more acceptable for the drug, initiating chronic therapy for the individual human patient using the drug, taking into account the relative benefits of the drug based on the results of the evaluation of safety, effectiveness, patient compliance and desirability of the drug and the placebo as compared to the patient population database; (ii) if safety, effectiveness, patient compliance and desirability are more acceptable for the placebo, initiating chronic therapy for the individual human patient using the placebo or a low risk, less costly alternative therapy; (iii) if safety, effectiveness, patient compliance and desirability are not acceptable for the drug and the placebo, discontinuing treatment or repeating steps (b)-(d) utilizing a second drug and placebo, if available; and thereafter if safety, effectiveness, patient compliance and desirability are more acceptable for the second drug, initiating chronic therapy for the individual human patient using the second drug.

13. The method of claim 12, further comprising assembling said patient population database from a plurality of cross-over single patient drug trials prior to conducting step a.

14. The method of claim 12, further comprising adding the results from the single patient drug trial of the individual human patient to the patient population database.

15. The method of claim 12, further comprising accumulating the information of step (b) via the use of objective testing methodologies selected from the group consisting of blood pressure, cholesterol, blood sugar, glycosylated hemoglobin and combinations of any of the foregoing.

16. The method of claim 12, further comprising including a questionnaire in said test, said questionnaire designed to elicit from said individual patient or caretaker information concerning the actual usage, safety, effectiveness and desirability of said drug and said second agent.

17. The method of claim 12, further comprising prescribing said drug for chronic therapy in said patient.

18. The method of claim 12, wherein said patient population database is stored on a computer.

19. The method of claim 18, wherein said computer database is accessible from a remote location.

20. The method according to claim 12, wherein said drug is selected from the group consisting of a drug for treating hyperkinetic behavior, cancer, schizophrenia, minimal brain dysfunction, mania, alzheimer's disease, attention deficit disorder (ADD), angina, congestive heart failure, cardiac arrhythmias, pain, metabolic disorders, endocrine disorders, obesity, neurologic disorders, immunologic diseases, eye disorders, ear disorders, sleep disorders, central nervous system disorders, urinary tract disorders, renal disorders, genito-urinary disorders, erectile dysfunction, podiatric disorders, chiropractic disorders, geriatric conditions, anti-asthmatic agents, dental agents, anti-epileptic agents, anti-psychotic agents, anti-depressants, cardiovascular agents, respiratory agents, neurological agents, antihypertensive agents, diabetic agents, steroidal and non-steroidal anti-inflammatory agents, opiates, narcotic and non-narcotic analgesics, hematologic agents, musculoskeletal agents, anti-anxiety agents, gastro-intestinal agents, dermatologic agents; and anti-allergy medications.

21. The method of claim 12, wherein said prescribability test kit comprises a supply of a drug therapy; a supply of placebo; and a questionnaire designed to elicit from said individual or caretaker information concerning the actual usage, safety, effectiveness and desirability of said drug therapy and said placebo.

22. The method of claims 12, further comprising prescribing said second drug for chronic treatment of said patient.

23. A method of optimizing clinical outcomes and providing optimized pharmacotherapy in an individual human patient for whom chronic drug therapy is contemplated, comprising: a) determining a first dose of a drug for treatment of an individual human patient for whom chronic drug therapy is contemplated, and a second dose of the same drug which may alternatively be useful for treatment of the individual human patient; b) conducting a single patient cross-over drug trial in the individual human patient via a dosability test utilizing a supply of the first dose of drug and a supply of the second dose of the same drug; and accumulating information concerning the safety, effectiveness, patient compliance and desirability of the first dose of drug, and the second dose of the same drug; c) evaluating whether safety, effectiveness, patient compliance and desirability are more acceptable for the first dose of drug than the second dose of the same drug; the second dose of drug than the first dose of the same drug; or neither the first dose of drug or the second dose of the same drug, by comparing the results of the single patient drug trial of the individual human patient with a previously assembled patient population database of information concerning the safety, effectiveness, patient compliance and desirability of the first dose of drug and the second dose of the same drug administered in a plurality of crossover single patient drug trials, to aid in the interpretation of the results for the new patient; and d) optimizing treatment for the new patient by taking one of the following actions: (i) if safety, effectiveness, patient compliance and desirability is more acceptable for the first dose of drug, initiating chronic therapy for the individual human patient using the first dose of drug, taking into account the relative benefits of each dose of drug based on the results of the evaluation of safety, effectiveness, patient compliance and desirability of said first dose of drug and said second dose of said same drug as compared to the patient population database, as well as the relative cost of the first dose of drug and the second dose of the same drug; (ii) if safety, effectiveness, patient compliance and desirability are more acceptable for the second dose of drug than the first dose of the same drug, initiating chronic therapy for the individual human patient using the second dose of drug; (iii) if safety, effectiveness, patient compliance and desirability are not more acceptable for either of the first dose of drug and the second dose of the same drug, discontinuing treatment or repeating steps (b)-(d) utilizing new first and second dose of the same drug or a first and a second dose of a second alternative drug, if available; and, thereafter, if safety, effectiveness, patient compliance and desirability are more acceptable for either the new or the first or second dose of the alternative drug, initiating chronic therapy for the individual human patient using that dose of the drug or second alterative drug.

24. The method of claim 23, further comprising assembling said patient population database from a plurality of cross-over single patient drug trials prior to conducting step a.

25. The method of claim 23, further comprising adding the results from the single patient drug trial of the individual human patient to the patient population database.

26. The method of claim 23, further comprising accumulating the information of step (b) via the use of objective testing methodologies selected from the group consisting of blood pressure, cholesterol, blood sugar, glycosylated hemoglobin and combinations of any of the foregoing.

27. The method of claim 23, further comprising including a questionnaire in said test, said questionnaire designed to elicit from said individual patient or caretaker information concerning the actual usage, safety, effectiveness and desirability of said drug and said second agent.

28. The method of claim 23, further comprising prescribing said first dose of drug for chronic therapy in said patient.

29. The method of claim 23, further comprising prescribing said second dose of drug for chronic therapy in said patient.

30. The method of claim 23, wherein said patient population database is stored on a computer.

31. The method of claim 30, wherein said computer database is accessible from a remote location.

32. The method according to claim 23, wherein said drug is selected from the group consisting of a drug for treating hyperkinetic behavior, cancer, schizophrenia, minimal brain dysfunction, mania, alzheimer's disease, attention deficit disorder (ADD), angina, congestive heart failure, cardiac arrhythmias, pain, metabolic disorders, endocrine disorders, obesity, neurologic disorders, immunologic diseases, eye disorders, ear disorders, sleep disorders, central nervous system disorders, urinary tract disorders, renal disorders, genito-urinary disorders, erectile dysfunction, podiatric disorders, chiropractic disorders, geriatric conditions, anti-asthmatic agents, dental agents, anti-epileptic agents, anti-psychotic agents, anti-depressants, cardiovascular agents, respiratory agents, neurological agents, antihypertensive agents, diabetic agents, steroidal and non-steroidal anti-inflammatory agents, opiates, narcotic and non-narcotic analgesics, hematologic agents, musculoskeletal agents, anti-anxiety agents, gastroin-testinal agents, dermatologic agents; and anti-allergy medications.

33. The method of claim 23, wherein said dosability test kit comprises a supply of a high dose of drug therapy; a supply of a low dose of drug therapy; and a questionnaire designed to elicit from said individual or caretaker information concerning the actual usage, safety, effectiveness and desirability of said high dose of drug therapy and said low dose of drug therapy.

34. The method of claim 23, further comprising prescribing said high dose of drug therapy.

35. The method of claim 23, further comprising prescribing said low dose of drug therapy.
